(19) United States
(12) Patent Application Publication
(10) Pub. No.: US 2008/0194598 A1
(43) Pub. Date: Aug. 14, 2008

(54) Pyrimidine Carboxylic Acid Derivatives and Use Thereof

(75) Inventors: Elisabeth Woltering, Hilden (DE); Arounarith Tuch, Lyon (FR); Elke Dittrich-Wengenroth, Wuppertal (DE); Axel Kretschmer, Wuppertal (DE); Lars Barfacker, Oberhausen (DE); Marcus Bauser, Wuppertal (DE); Peter Ellinghaus, Melle (DE); Klemens Lustig, Wuppertal (DE); Elisabeth Pook, Wuppertal (DE); Olaf Weber, Wulfrath (DE)

Correspondence Address: Bayer Health Care LLC, 400 Morgan Lane, West Haven, CT, 06516, US

(73) Assignee: Bayer HealthCare AG, Leverkusen (DE)

(21) Appl. No.: 11/886,289

(22) PCT Filed: Mar. 7, 2006
(86) PCT No.: PCT/EP06/02054

§ 371 Date: Mar. 10, 2008

(30) Foreign Application Priority Data
Mar. 12, 2005 (DE) 10 2005 011 447.4
Jun. 11, 2005 (DE) 10 2005 027 150.2

(51) Int. Cl. A61K 31/513; C07D 239/34; C07D 239/26; A61K 31/505; A61P 9/10

(52) U.S. Cl. 514/269; 544/335; 514/256; 544/319

(57) ABSTRACT

The present application relates to pyrimidinecarboxylic acid derivatives, to processes for their preparation, to their use for the treatment and/or prophylaxis of diseases and their use for preparing medicaments for the treatment and/or prophylaxis of diseases, preferably for the treatment and/or prevention of cardiovascular disorders, in particular dyslipidaemias and arteriosclerosis.

The present invention relates to pyrimidinecarboxylic acid derivatives, to processes for their preparation, to their use for the treatment and/or prophylaxis of diseases and to their use for preparing medicaments for the treatment and/or prophylaxis of diseases, preferably for the treatment and/or prevention of cardiovascular diseases, in particular dyslipidaemias and arteriosclerosis.

In spite of many successful therapies, cardiovascular disorders remain a serious public health problem. Treatment with statins, which inhibit HMG-CoA reductase, very successfully lowers both LDL cholesterol (LDL-C) plasma concentrations and the mortality of patients at risk; however, convincing treatment strategies for the therapy of patients having an unfavourable HDL-C/LDL-C ratio and/or hypertriglyceridaemia are still not available to date.

Currently, in addition to niacin, fibrates are the only therapy option for patients of these risk groups. They lower elevated triglyceride levels by 20-50%, reduce LDL-C by 10-15%, change the LDL particle size of atherogenic LDL of low density to less atherogenic LDL of normal density and increase the HDL concentration by 10-15%.

Fibrates act as weak agonists of the peroxysome-proliferator-activated receptor (PPAR)-alpha (Nature 1990, 347, 645-50). PPAR-alpha is a nuclear receptor which regulates the expression of target genes by binding to DNA sequences in the promoter range of these genes [also referred to as PPAR response elements (PPRE)]. PPREs have been identified in a number of genes coding for proteins which regulate lipid metabolism. PPAR-alpha is highly expressed in the liver, and its activation leads inter alia to lower VLDL production/secretion and reduced apolipoprotein CIII (ApoCIII) synthesis. In contrast, the synthesis of apolipoprotein Al (ApoAl) is increased.

A disadvantage of fibrates which have hitherto been approved is that their interaction with the receptor is only weak (EC₅₀ in the μM range), which in turn is responsible for the relatively small pharmacological effects described above.

It was an object of the present invention to provide novel compounds suitable for use as PPAR-alpha modulators for the treatment and/or prevention of in particular cardiovascular disorders.

Ethyl 4-(2-methylphenoxy)-2-phenylpyrimidine-5-carboxylate and the corresponding carboxylic acid are described in WO 02/42280 as synthesis intermediates; a pharmacological activity of these compounds is not reported in this publication. U.S. Pat. No. 3,759,922, U.S. Pat. No. 3,850,931 and J. Heterocyclic Chem. 9 (6), 1347-54 (1972) describe certain 4-phenoxy-2-phenylpyrimidine-5-carboxylic acid derivatives as synthesis intermediates, some of which have a mydriatic or the activity of the central nervous system suppressing effect. WO 02/076438 claims inter alia pyrimidine derivatives as Flt 1 ligands for the treatment of cancer and various other disorders.

The present invention provides compounds of the general formula (I)

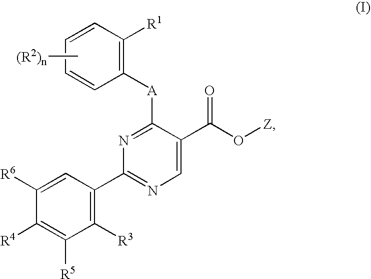

in which
A represents CH₂ or O,
R¹ represents halogen, cyano or (C₁-C₄)-alkyl,
R² represents a substituent selected from the group consisting of halogen, cyano, (C₁-C₆)-alkyl and (C₁-C₆)-alkoxy in which alkyl and alkoxy for their part may be mono- or polysubstituted by fluorine, or represents a group of the formula —NR⁷—C(═O)—R⁸, in which
   R⁷ represents hydrogen or (C₁-C₆)-alkyl
   and
   R⁸ represents hydrogen, (C₁-C₆)-alkyl or (C₁-C₆)-alkoxy,
n represents the number 0, 1, 2 or 3,
   where in the case that the substituent R² is present more than once its meanings may be identical or different,
R³ represents hydrogen, fluorine or chlorine,
R⁴ represents hydrogen, halogen, nitro, cyano, amino, trifluoromethyl, (C₁-C₄)-alkyl or (C₁-C₄)-alkoxy,
R⁵ and R⁶ are identical or different and independently of one another represent hydrogen, halogen, nitro, cyano, (C₁-C₆)-alkyl or (C₁-C₆)-alkoxy in which alkyl and alkoxy for their part may be mono- or polysubstituted by fluorine, represent amino, mono- or di-(C₁-C₆)-alkylamino, a 4- to 7-membered heterocycle which is attached via an N atom, or represent a group of the formula —NR⁹—C(═O)—R¹⁰, in which
   R⁹ represents hydrogen or (C₁-C₆)-alkyl
   and
   R¹⁰ represents hydrogen, (C₁-C₆)-alkyl or (C₁-C₆)-alkoxy,
      and
Z represents hydrogen or (C₁-C₄)-alkyl,
   and their salts, solvates and solvates of the salts,
   for the treatment and/or prophylaxis of diseases, in particular the use of these compounds for preparing a medicament for the treatment and/or prophylaxis of cardiovascular disorders.

Most of the abovementioned compounds are novel, but some are also known from the literature [see WO 02/42280 and also the compounds having the Chemical Abstracts Registry Nos. 477859-49-7, 477859-47-5, 477854-82-3 and 477854-79-8]. However, a therapeutic application of known compounds has hitherto not been described. For the first time, this is the case in the context of the present invention.

The present invention also provides compounds of the general formula (I) in which
A represents CH₂ or O ,
R¹ represents halogen, cyano or (C₁-C₄)-alkyl,
R² represents a substituent selected from the group consisting of halogen, cyano, (C₁-C₆)-alkyl and (C₁-C₆)-alkoxy in which alkyl and alkoxy for their part may be mono- or polysubstituted by fluorine, or represents a group of the formula —NR⁷—C(═O)—R⁸, in which
   R⁷ represents hydrogen or (C₁-C₆)-alkyl
   and
   R⁸ represents hydrogen, (C₁-C₆)-alkyl or (C₁-C₆)-alkoxy,
n represents the number 0, 1, 2 or 3,
   where in the case that the substituent R² is present more than once its meanings may be identical or different,
R³ represents hydrogen, fluorine or chlorine,
R⁴ represents hydrogen, halogen, nitro, cyano, amino, trifluoromethyl, (C₁-C₄)-alkyl or (C₁-C₄)-alkoxy,
R⁵ and R⁶ are identical or different and independently of one another represent hydrogen, halogen, nitro, cyano, (C₁-C₆)-alkyl or (C₁-C₆)-alkoxy in which alkyl and alkoxy for their part may be mono- or polysubstituted by fluorine, represent amino, mono- or di-(C₁-C₆)-alkylamino, a 4- to 7-membered heterocycle which is attached via an N atom, or represent a group of the formula —NR⁹—C(═O)—R¹⁰, in which
   R⁹ represents hydrogen or (C₁-C₆)-alkyl
   and
   R¹⁰ represents hydrogen, (C₁-C₆)-alkyl or (C₁-C₆)-alkoxy,
      and
Z represents hydrogen or (C₁-C₄)-alkyl,
   and their salts, solvates and solvates of the salts,
   except for the compounds ethyl 4-(2-methylphenoxy)-2-phenylpyrimidine-5-carboxylate, 4-(2-methylphenoxy)-2-phenylpyrimidine-5-carboxylic acid, ethyl 4-(2,3-dimethylphenoxy)-2-phenylpyrimidine-5-carboxylate, 4-(2,3-dimethylphenoxy)-2-phenylpyrimidine-5-carboxylic acid, ethyl 2-phenyl-4-(2,4,5-trichlorophenoxy)pyrimidine-5-carboxylate and 2-phenyl-4-(2,4,5-trichlorophenoxy)pyrimidine-5-carboxylic acid.

Compounds according to the invention are the compounds of the formula (I) and their salts, solvates and solvates of the salts, the compounds, comprised by formula (I), of the formulae mentioned below and their salts, solvates and solvates of the salts and the compounds, comprised by the formula (I), mentioned below as embodiments and their salts, solvates and solvates of the salts if the compounds, comprised by formula (I), mentioned below are not already salts, solvates and solvates of the salts.

Depending on their structure, the compounds according to the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform components in a known manner.

If the compounds according to the invention can be present in tautomeric forms, the present invention comprises all tautomeric forms.

In the context of the present invention, preferred salts are physiologically acceptable salts of the compounds according to the invention. The invention also comprises salts which for their part are not suitable for pharmaceutical applications, but which can be used, for example, for isolating or purifying the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds according to the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds according to the invention. The term “prodrugs” includes compounds which for their part may be biologically active or inactive but which, during the time they spend in the body, are converted into compounds according to the invention (for example metabolically or hydrolytically).

In the context of the present invention, unless specified differently, the substituents have the following meanings:

In the context of the invention, (C₁-C₆)-alkyl and (C₁-C₄)-alkyl represent a straight-chain or branched alkyl radical having 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl, n-butyl, iso-butyl, sec-butyl, tert-butyl, 1-ethylpropyl, n-pentyl and n-hexyl.

In the context of the invention, (C₁-C₆)-alkoxy and (C₁-C₄)-alkoxy represent a straight-chain or branched alkoxy radical having 1 to 6 and 1 to 4 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy and tert-butoxy.

In the context of the invention, mono-(C₁-C₆)-alkylamino and mono-(C₁-C₄)-alkylamino represent an amino group having a straight-chain or branched alkyl substituent which has 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino and tert-butylamino.

In the context of the invention, di-(C₁-C₆)-alkylamino and di-(C₁-C₄)-alkylamino represent an amino group having two identical or different straight-chain or branched alkyl substituents which have in each case 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, a 4- to 7-membered heterocycle represents a saturated or partially unsaturated heterocycle having 4 to 7 ring atoms which contains a ring nitrogen atom and is attached via this ring nitrogen atom and may contain a further heteroatom from the group consisting of N, O, S, SO and SO₂. Preference is given to a 5- or 6-membered saturated N-attached heterocycle which may contain a further heteroatom from the group consisting of N, O and S. The following radicals may be mentioned by way of example: pyrrolidinyl, pyrrolinyl, thiazolidinyl, piperidinyl, piperazinyl, morpholinyl, thiomorpholinyl, azepinyl and 1,4-diazepinyl. Preference is given to piperidinyl, piperazinyl, morpholinyl, thiomorpholinyl and pyrrolidinyl.

In the context of the invention, halogen includes fluorine, chlorine, bromine and iodine. Preference is given to chlorine or fluorine.

If radicals in the compounds according to the invention are substituted, the radicals can, unless specified otherwise, be mono- or polysubstituted. In the context of the present invention, the meanings of radicals which occur more than once are independent of one another. Substitution with one, two or three identical or different substituents is preferred. Very particular preference is given to substitution with one substituent.

In the context of the present invention, preference is given to compounds of the formula (I) in which
A represents CH₂ or O,
R¹ represents halogen, cyano or (C₁-C₄)-alkyl,
R² represents a substituent selected from the group consisting of halogen, cyano, (C₁-C₄)-alkyl and (C₁-C₄)-alkoxy in which alkyl and alkoxy for their part may be mono- or polysubstituted by fluorine,
n represents the number 0, 1, 2 or 3,
   where in the case that the substituent R² is present more than once its meanings may be identical or different,
R³ represents hydrogen, fluorine or chlorine,
R⁴ represents hydrogen, halogen, cyano, trifluoromethyl, (C₁-C₄)-alkyl or (C₁-C₄)-alkoxy,
R⁵ and R⁶ are identical or different and independently of one another represent hydrogen, halogen, nitro, cyano, (C₁-C₄)-alkyl or (C₁-C₄)-alkoxy in which alkyl and alkoxy for their part may be mono- or polysubstituted by fluorine, or represent amino, mono- or di-(C₁-C₄)-alkyl-amino,
   and
Z represents hydrogen, methyl or ethyl,
   where at least one of the radicals R³, R⁴, R⁵ and R⁶ is different from hydrogen,
   and their salts, solvates and solvates of the salts.

In the context of the present invention, particular preference is given to compounds of the formula (I) in which
A represents O,
R¹ represents fluorine, chlorine, bromine, cyano or methyl,
R² represents a substituent selected from the group consisting of fluorine, chlorine, bromine, cyano, (C₁-C₄)-alkyl, trifluoromethyl, (C₁-C₄)-alkoxy and trifluoromethoxy,
n represents the number 0, 1, 2 or 3,
   where in the case that the substituent R² is present more than once its meanings may be identical or different,
R³ represents hydrogen or fluorine,
R⁴ represents hydrogen, fluorine, chlorine, trifluoromethyl or methyl,
R⁵ and R⁶ are identical or different and independently of one another represent hydrogen, fluorine, chlorine, bromine, nitro, cyano, (C₁-C₄)-alkyl, trifluoromethyl, (C₁-C₄)-alkoxy, trifluoromethoxy or amino,
   and
Z represents hydrogen,
   where at least one of the radicals R³, R⁴, R⁵ and R⁶ is different from hydrogen, and their salts, solvates and solvates of the salts.

The individual radical definitions given in the respective combinations or preferred combinations of radicals may, independently of the particular given combination of radicals, also be replaced by any radical definitions of other combinations.

Very particular preference is given to combinations of two or more of the preferred ranges mentioned above.

The invention furthermore provides a process for preparing the compounds of the formula (I) according to the invention in which A represents O, characterized in that the compounds of the formula (II)

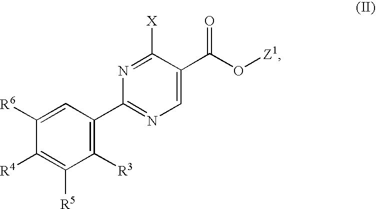

in which R³, R⁴, R⁵ and R⁶ are each as defined above and
Z¹ represents (C₁-C₄)-alkyl
   and
X represents a suitable leaving group, such as, for example, halogen, in particular chlorine,
   are reacted in an inert solvent in the presence of a base with a compound of the formula (III)

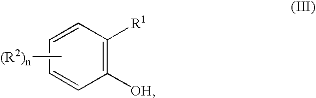

in which R¹, R² and n are each as defined above,
to give compounds of the formula (I-A)

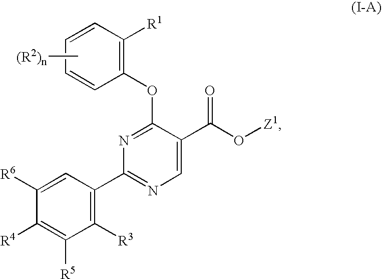

in which R¹, R², R³, R⁴, R⁵, R⁶, Z¹ and n are each as defined above,
and these are converted by basic or acidic hydrolysis into the carboxylic acids of the formula (I-B)

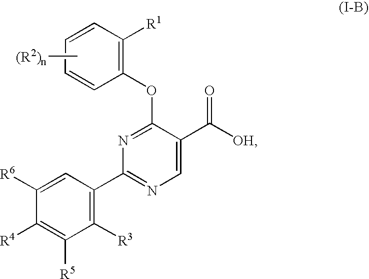

in which R¹, R², R³, R⁴, R⁵, R⁶ and n are each as defined above
and the compounds of the formulae (I-A) and (I-B) are, if appropriate, converted into their solvates, salts and/or solvates of the salts using the appropriate (i) solvents and/or (ii) bases or acids.

The compounds of the formula (II) can be prepared by initially reacting nitrile of the formula (IV)

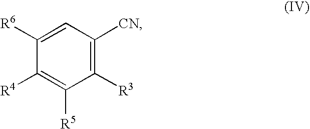

in which R³, R⁴, R⁵ and R⁶ are each as defined above in an inert solvent with ammonium chloride in the presence of trimethylaluminium to give amidines of the formula (V)

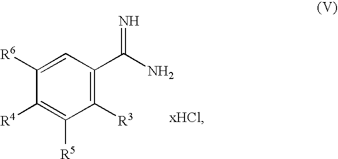

in which R³, R⁴, R⁵ and R⁶ are each as defined above,
then condensing in the presence of a base with a compound of the formula (VI)

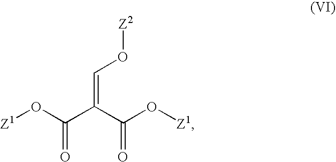

in which Z¹ is as defined above and
Z² represents methyl or ethyl,
   to give compounds of the formula (VII)

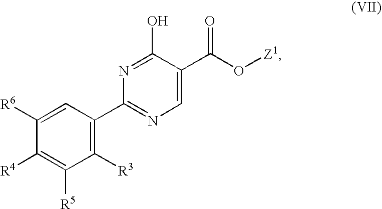

in which R³, R⁴, R⁵, R⁶ and Z¹ are each as defined above,
and then converting these in an inert solvent with the aid of a suitable halogenating agent, such as, for example, thionyl chloride, into the compounds of the formula (II).

The compounds of the formulae (III), (IV) and (VI) are commercially available, known from the literature or can be prepared analogously to processes known from the literature.

Inert solvents for the process step (II)+(III)→(I-A) are, for example, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as dimethylformamide, dimethyl sulphoxide, N,N′-dimethylpropyleneurea (DMPU), N-methylpyrrolidinone (NMP), pyridine or acetonitrile. It is also possible to use mixtures of the solvents mentioned. Preference is given to dimethylformamide or acetonitrile. Suitable bases for process step (II)+(III)→(I-A) are customary inorganic bases. These include in particular alkali metal hydroxides, such as, for example, lithium hydroxide, sodium hydroxide or potassium hydroxide, alkali metal carbonates or alkaline earth metal carbonates, such as lithium carbonate, sodium carbonate, potassium carbonate, calcium carbonate or caesium carbonate, or alkali metal hydrides, such as sodium or potassium hydride. Preference is given to potassium carbonate or sodium hydride.

Here, the base is employed in an amount of from 1 to 3 mol, preferably in an amount of from 1.2 to 2 mol, per mole of the compound of the formula (III). The reaction is generally carried out in a temperature range of from 0° C. to +100° C., preferably from +20° C. to +60° C. The reaction can be carried out at atmospheric, elevated or at reduced pressure (for example from 0.5 to 5 bar). In general, the reaction is carried out at atmospheric pressure.

The hydrolysis of the carboxylic acid in process steps (I-A)→(I-B) and (I-C)→(I-D) is carried out by customary methods by treating the esters in inert solvents with bases, where the salts initially formed are converted by treatment with acid into the free carboxylic acids. In the case of the tert-butyl esters, the ester cleavage is preferably carried out using acids.

Suitable inert solvents for the hydrolysis of the carboxylic esters are water or organic solvents customary for ester cleavage. These preferably include alcohols, such as methanol, ethanol, n-propanol, isopropanol, n-butanol or tert-butanol, or ethers, such as diethyl ether, tetrahydrofuran, dioxane or glycol dimethyl ether, or other solvents, such as acetone, acetonitrile, dichloromethane, dimethylformamide or dimethyl sulphoxide. It is also possible to use mixtures of the solvents mentioned. In the case of a basic ester hydrolysis, preference is given to using mixtures of water with dioxane, tetrahydrofuran, methanol and/or ethanol. In the case of the reaction with trifluoroacetic acid, preference is given to using dichloromethane, and in the case of the reaction with hydrogen chloride, preference is given to using tetrahydrofuran, diethyl ether, dioxane or water.

Suitable bases for the ester hydrolysis are the customary inorganic bases. These preferably include alkali metal hydroxides or alkaline earth metal hydroxides such as, for example, sodium hydroxide, lithium hydroxide, potassium hydroxide or barium hydroxide, or alkali metal carbonates or alkaline earth metal carbonates, such as sodium carbonate, potassium carbonate or calcium carbonate. Particular preference is given to using sodium hydroxide or lithium hydroxide.

Suitable acids for the ester cleavage are, in general, sulphuric acid, hydrogen chloride/hydrochloric acid, hydrogen bromide/hydrobromic acid, phosphoric acid, acetic acid, trifluoroacetic acid, toluenesulphonic acid, methanesulphonic acid or trifluoromethanesulphonic acid or mixtures thereof, if appropriate with addition of water. Preference is given to hydrogen chloride or trifluoroacetic acid in the case of the tert-butyl esters and to hydrochloric acid in the case of the methyl esters.

The ester cleavage is generally carried out in a temperature range of from 0° C. to +100° C., preferably from 0° C. to +40° C. The reaction can be carried out at atmospheric, elevated or at reduced pressure (for example from 0.5 to 5 bar). In general, the reaction is carried out at atmospheric pressure.

Suitable inert solvents for the process step (IV)→(V) are, for example, halogenated hydrocarbons, such as dichloromethane, trichloromethane, carbon tetrachloride, 1,2-dichloroethane, trichloroethylene or chlorobenzene, or hydrocarbons, such as benzene, xylene, toluene, hexane, cyclohexane or mineral oil fractions. It is also possible to use mixtures of the solvents mentioned. Preference is given to toluene.

The reaction partners ammonium chloride and trimethylaluminium used in the process step (IV)→(V) are in each case employed in an amount of from 2 to 4 mol, preferably in an amount of from 2 to 3 mol, per mole of the compound of the formula (IV). The reaction is generally carried out in a temperature range of from +20° C. to +150° C., preferably from +80° C. to +120° C. The reaction can be carried out at atmospheric, elevated or at reduced pressure (for example from 0.5 to 5 bar). In general, the reaction is carried out at atmospheric pressure.

Suitable inert solvents for the process step (V)+(VI)→(VII) are, for example, alcohols, such as methanol, ethanol, n-propanol, isopropanol, n-butanol or tert-butanol, or ethers, such as diethyl ether, dioxane, tetrahydrofuran, glycol dimethyl ether or diethylene glycol dimethyl ether. It is also possible to use mixtures of the solvents mentioned. Preference is given to ethanol.

Suitable bases for the process step (V)+(VI)→(VII) are in particular alkali metal alkoxides, such as sodium methoxide or potassium methoxide, sodium ethoxide or potassium ethoxide or potassium tert-butoxide. Preference is given to sodium ethoxide.

Here, the base is employed in an amount of from 2 to 3 mol, preferably in an amount of from 2 to 2.5 mol, per mole of the compound of the formula (V). The reaction is generally carried out in a temperature range of from +20° C. to +100° C., preferably from +50° C. to +80° C. The reaction can be carried out at atmospheric, elevated or at reduced pressure (for example from 0.5 to 5 bar). In general, the reaction is carried out at atmospheric pressure.

The halogenation and process step (VII)→(II) is preferably carried out with the aid of thionyl chloride or using para-toluenesulphonyl chloride or methanesulphonyl chloride, the latter in each case in the presence of a tertiary amine, such as, for example, triethylamine, N-methylmorpholine, N-methylpiperidine or N,N-diisopropylethylamine.

Suitable inert solvents for the process step (VII)→(II) are, for example, halogenated hydrocarbons, such as dichloromethane, trichloromethane, carbon tetrachloride, trichloroethane, tetrachloroethane, 1,2-dichloroethane or trichloroethylene, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as dimethylformamide or dimethyl sulphoxide. It is also possible to use mixtures of the solvents mentioned. Preference is given to dimethylformamide and dichloromethane.

The reaction is generally carried out in a temperature range of from 0° C. to +60° C., preferably from 0° C. to +30° C. The reaction can be carried out at atmospheric, elevated or at reduced pressure (for example from 0.5 to 5 bar). In general, the reaction is carried out at atmospheric pressure.

The invention furthermore provides a process for preparing the compounds of the formula (I) according to the invention in which A represents CH₂, characterized in that either

[A] compounds of the formula (VIII)

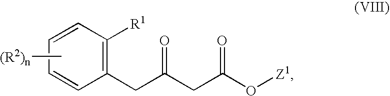

in which R¹, R² and n are each as defined above and
Z¹ represents (C₁-C₄)-alkyl,
are reacted with a compound of the formula (IX)

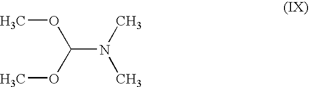

to give compounds of the formula (X)

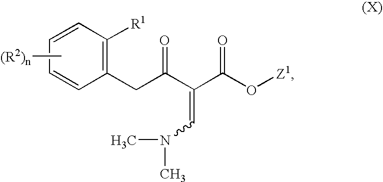

in which R¹, R², n and Z¹ are each as defined above
and then reacted in an inert solvent in the presence of a base with an amidine of the formula (V)

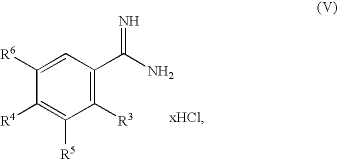

in which R³, R⁴, R⁵ and R⁶ are each as defined above,
to give compounds of the formula (I-C)

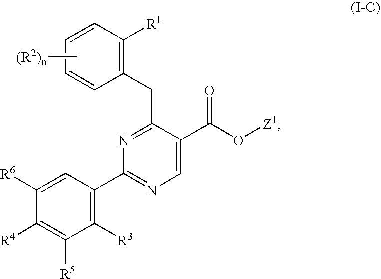

in which R¹, R², R³, R⁴, R⁵, R⁶, Z¹ and n are each as defined above
   or

[B] compounds of the formula (XI)

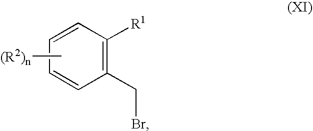

in which R¹, R² and n are each as defined above
are converted into the organotin compounds of the formula (XII)

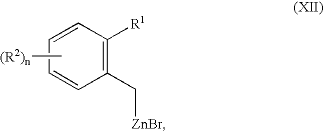

in which R¹, R² and n are each as defined above
and subsequently coupled in an inert solvent in the presence of a suitable palladium catalyst with a compound of the formula (II)

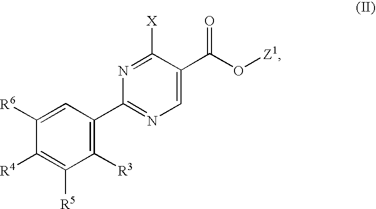

in which R³, R⁴, R⁵ and R⁶ are each as defined above and
Z¹ represents (C₁-C₄)-alkyl
and
X represents a suitable leaving group, such as, for example, halogen, in particular chlorine,
to give compounds of the formula (I-C)

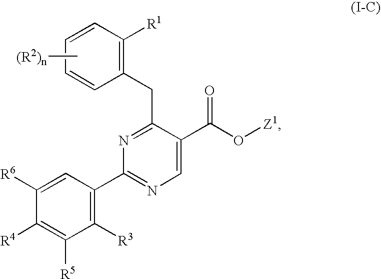

in which R¹, R², R³, R⁴, R⁵, R⁶, Z¹ and n are each as defined above
   and the resulting compounds of the formula (I-C) are converted by basic or acidic hydrolysis into the carboxylic acids of the formula (I-D)

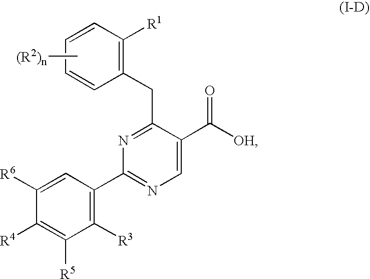

in which R¹, R², R³, R⁴, R⁵, R⁶ and n are each as defined above and the compounds of the formulae (I-C) and (I-D) are, if appropriate, converted into their solvates, salts and/or solvates of the salts using the appropriate (i) solvents and/or (ii) bases or acids.

The compounds of the formulae (VIII), (IX) and (XI) are commercially available, known from the literature or can be prepared analogously to processes known from the literature. The compounds of the formulae (II) and (V) can be prepared as described above.

The preparation of the compounds according to the invention can be illustrated by the synthesis schemes below:

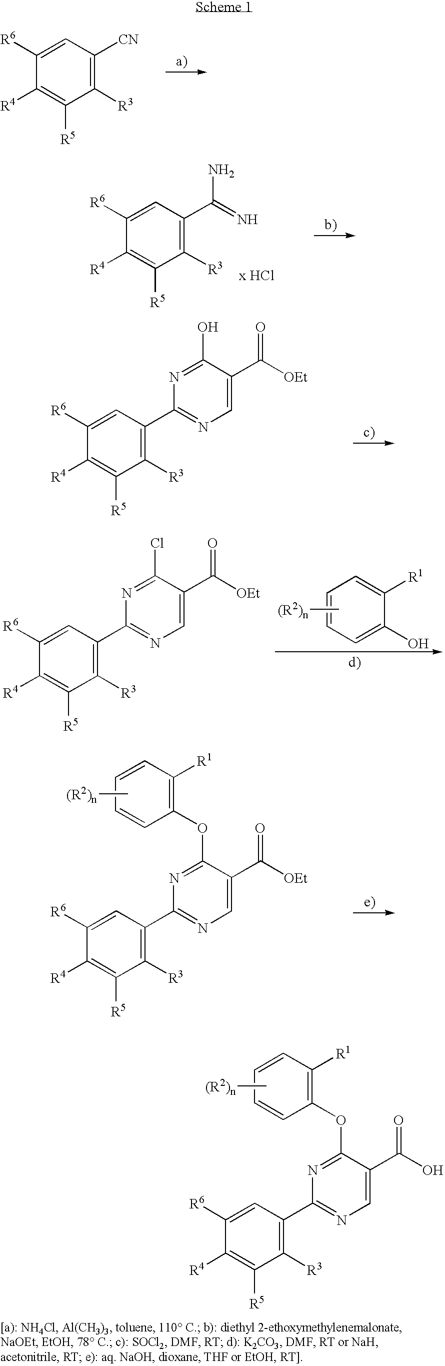

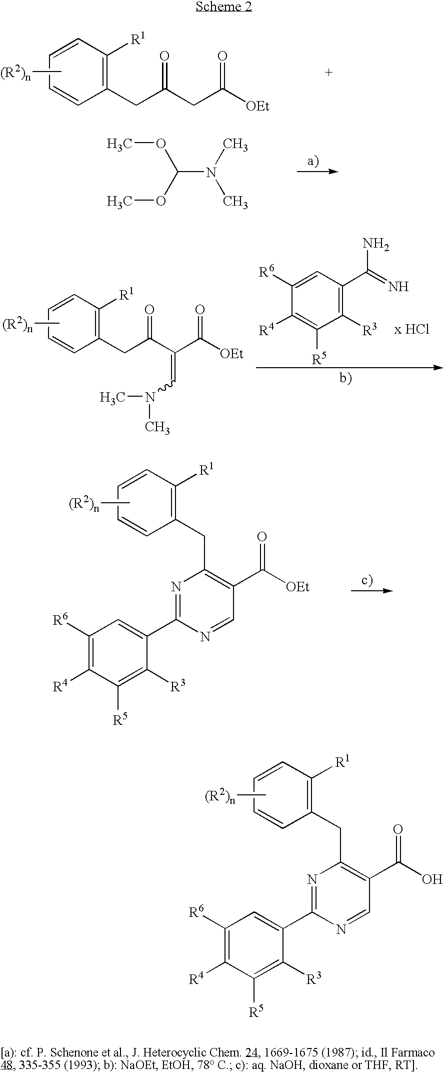

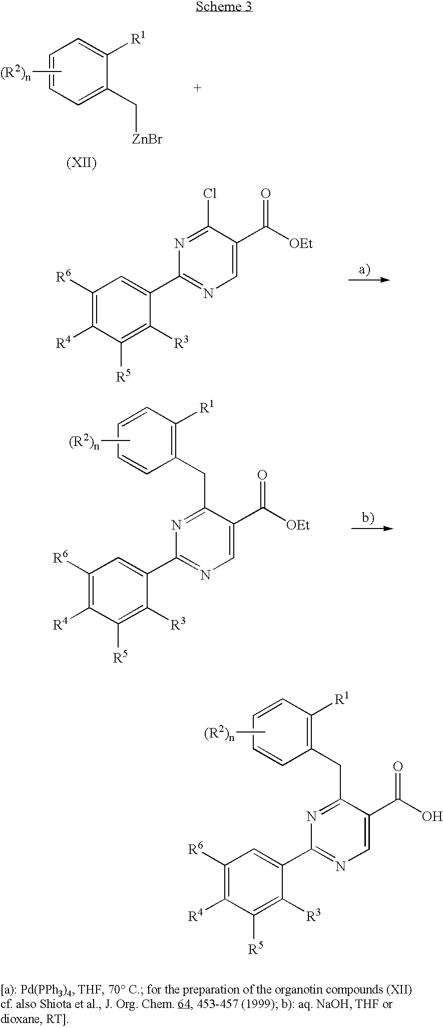

The compounds according to the invention have useful pharmacological properties and can be used the prevention and treatment of disorders in humans and animals.

The compounds according to the invention are highly effective PPAR-alpha modulators and as such are suitable in particular for the primary and/or secondary prevention and treatment of cardiovascular disorders caused by disturbances in fatty acid and glucose metabolism. Such disorders include dislipidaemias (hypercholesterolaemia, hypertriglyceridaemia, elevated concentrations of postprandial plasma triglycerides, hypoalphalipoproteinaemia, combined hyperlipidaemias), arteriosclerosis and metabolic disorders (metabolic syndrome, hyperglycaemia, insulin-dependent diabetes, non-insulin-dependent diabetes, gestation diabetes, hyperinsulinaemia, insulin resistance, glucose intolerance, obesity (adipositas) and late sequelae of diabetes, such as retinopathy, nephropathy and neuropathy).

Further independent risk factors for cardiovascular disorders which can be treated by the compounds according to the invention are high blood pressure, ischaemia, myocardial infaction, angina pectoris, cardiac insufficiency, myocardial insufficiency, restenosis, elevated levels of fibrinogen and of LDL of low density and also elevated concentrations of plasminogen activator inhibitor 1 (PAI-1).

In addition, the compounds according to the invention can also be used for the treatment and/or prevention of micro- and macrovascular damage (vasculitis), reperfusion damage, arterial and venous thromboses, oedema, cancerous disorders (skin cancer, liposarcomas, carcinomas of the gastrointestinal tract, of the liver, of the pancreas, of the lung, of the kidney, of the urethra, of the prostate and of the genital tract), of disorders of the central nervous system and neurodegenerative disorders (strokes, Alzheimer's disease, Parkinson's disease, dementia, epilepsy, depressions, multiple sclerosis), of inflammatory disorders, immune disorders (Crohn's disease, ulcerative colitis, lupus erythematodes, rheumatoid arthritis, asthma), renal disorders (glomerulonephritis), disorders of the thyroid gland, disorders of the pancreas (pancreatitis), fibrosis of the liver, skin disorders (psoriasis, acne, eczema, neurodermitis, dermatitis, keratitis, formation of scars, formation of warts, frostbites), viral diseases (HPV, HCMV, HIV), kachexia, osteoporose, gout, incontinence, and also for wound healing and angiogenesis.

The activity of the compounds according to the invention can be examined, for example, in vitro by the transactivation assay described in the experimental section.

The in vivo activity of the compounds according to the invention can be examined, for example, by the tests described in the experimental section.

The present invention furthermore provides the use of the compounds according to the invention for the treatment and/or prevention of disorders, in particular the disorders mentioned above.

The present invention also provides the use of the compounds according to the invention for preparing a medicament for the treatment and/or prevention of disorders, in particular the disorders mentioned above.

The present invention also provides a method for the treatment and/or prevention of disorders, in particular the disorders mentioned above, using an effective amount of at least one compound according to the invention.

The compounds according to the invention can be used alone or, if required, in combination with other active compounds. The present invention furthermore provides medicaments comprising at least one compound according to the invention and one or more further active compounds, in particlar for the treatment and/or prevention of the disorders mentioned above.

Suitable active compounds for combinations are, by way of example and by way of preference: substances which modulate lipid metabolism, antidiabetics, hypertensive agents, perfusion-enhancing and/or antithrombotic agents and also antioxidants, chemokine receptor antagonists, p38-kinase inhibitor, NPY agonists, orexin agonists, anorectics, PAF-AH inhibitors, antiphlogistics (COX inhibitors, LTB₄-receptor antagonists), analgesics (aspirin), antidepressants and other psychopharmaceuticals.

The present invention provides in particular combinations comprising at least one of the compounds according to the invention and at least one lipid metabolism-modulating active compound, an antidiabetic, a hypertensive compound and/or antithrombotic agent.

Preferably, the compounds according to the invention can be combined with one or more
lipid metabolism-modulating active compounds, by way of example and by way of preference from the group of the HMG-CoA reductase inhibitors, inhibitors of HMG-CoA reductase expression, squalene synthesis inhibitors, ACAT inhibitors, LDL receptor inductors, cholesterol absorption inhibitors, polymeric bile acid adsorbers, bile acid reabsorption inhibitors, MTP inhibitors, lipase inhibitors, LpL activators, fibrates, niacin, CETP inhibitors, PPAR-γ and/or PPAR-δ agonists, RXR modulators, FXR modulators, LXR modulators, thyroid hormones and/or thyroid mimetics, ATP citrate lyase inhibitors, Lp(a) antagonists, cannabinoid receptor 1 antagonists, leptin receptor agonists, bombesin receptor agonists, histamine receptor agonists and the antioxidants/radical scavengers,
antidiabetics mentioned in the Rote Liste 2004/II, chapter 12, and also, by way of example and by way of preference, those from the group of the sulphonylureas, biguanides, meglitinide derivatives, glucosidase inhibitors, oxadiazolidinones, thiazolidinediones, GLP 1 receptor agonists, glucagon antagonists, insulin sensitizers, CCK 1 receptor agonists, leptin receptor agonists, inhibitors of liver enzymes involved in the stimulation of gluconeogenesis and/or glycogenolysis, modulators of glucose uptake and also potassium channel openers, such as, for example, those disclosed in WO 97/26265 and WO 99/03861,
hypotensives, by way of example and by way of preference from the group of the calcium antagonists, angiotensin All antagonists, ACE inhibitors, beta-receptor blockers, alpha-receptor blockers, diuretics, phosphodiesterase inhibitors, sGC stimulators, cGMP level elevating substances, aldosterone antagonists, mineralocorticoid receptor antagonists, ECE inhibitors and the vasopeptidase inhibitors, and/or
antithrombotic agents, by way of example and by way of preference from the group of the platelet aggregation inhibitors or the anticoagulants.

Lipid metabolism-modifying active compounds are to be understood as meaning, preferably, compounds from the group of the HMG-CoA reductase inhibitors, squalene synthesis inhibitors, ACAT inhibitors, cholesterol absorptions inhibitors, MTP inhibitors, lipase inhibitors, thyroid hormones and/or thyroid mimetics, niacin receptor agonists, CETP inhibitors, PPAR-gamma agonists, PPAR-delta agonists, polymeric bile acid adsorbers, bile acid reabsorption inhibitors, antioxidants/radical scavengers and also the cannabinoid receptor 1 antagonists.

In a preferred embodiment of the invention, the compounds according to the invention are administered in combination with an HMG-CoA reductase inhibitor from the class of the statins, such as, by way of example and by way of preference, lovastatin, simvastatin, pravastatin, fluvastatin, atorvastatin, rosuvastatin, cerivastatin or pitavastatin.

In a preferred embodiment of the invention, the compounds according to the invention are administered in combination with a squalene synthesis inhibitor, such as, by way of example and by way of preference, BMS-188494 or TAK-475.

In a preferred embodiment of the invention, the compounds according to the invention are administered in combination with an ACAT inhibitor, such as, by way of example and by way of preference, melinamide, pactimibe, eflucimibe or SMP-797.

In a preferred embodiment of the invention, the compounds according to the invenetion are administered in combination with a cholesterol absorption inhibitor, such as, by way of example and by way of preference, ezetimibe, tiqueside or pamaqueside.

In a preferred embodiment of the invention, the compounds according to the invention are administered in combination with an MTP inhibitor, such as, by way of example and by way of preference, implitapide or JTT-130.

In a preferred embodiment of the invention, the compounds according to the invention are administered in combination with a lipase inhibitor, such as by way of example and by way of preference, orlistat.

In a preferred embodiment of the invention, the compounds according to the invention are administered in combination with a thyroid hormone and/or thyroid mimetic, such as, by way of example and by way of preference, D-thyroxine or 3,5,3′-triiodothyronine (T3).

In a preferred embodiment of the invention, the compounds according to the invention are administered in combination with an agonist of the niacin receptor, such as, by way of example and by way of preference, niacin, acipimox, acifran or radecol.

In a preferred embodiment of the invention, the compounds according to the invention are administered in combination with a CETP inhibitor, such as, by way of example and by way of preference, torcetrapib, JTT-705 or CETP vaccine (Avant).

In a preferred embodiment of the invention, the compounds according to the invention are administered in combination with a PPAR-gamma agonist, such as, by way of example and by way of preference, pioglitazone or rosiglitazone.

In a preferred embodiment of the invention, the compounds according to the invention are administered in combination with a PPAR-delta agonist, such as, by way of example and by way of preference, GW-501516.

In a preferred embodiment of the invention, the compounds according to the invention are administered in combination with a polymeric bile acid adsorber, such as, by way of example and by way of preference, cholestyramine, colestipol, colesolvam, CholestaGel or colestimide.

In a preferred embodiment of the invention, the compounds according to the invention are administered in combination with a bile acid reabsorption inhibitor, such as, by way of example and by way of preference, ASBT (=IBAT) inhibitors, such as, for example, AZD-7806, S-8921, AK-105, BARI-1741, SC-435 or SC-635.

In a preferred embodiment of the invention, the compounds according to the invention are administered in combination with a antioxidant/radical scavenger, such as, by way of example and by way of preference, probucol, AGI-1067, BO-653 or AEOL-10150.

In a preferred embodiment of the invention, the compounds according to the invention are administered in combination with a cannabinoid receptor 1 antagonist, such as, by way of example and by way of preference, rimonabant or SR-147778.

Antidiabetics are to be understood as meaning, preferably, insulin and insulin derivatives, and also orally effective hypoglycaemic acid compounds. Here, insulin and insulin derivatives include both insulins of animal, human or biotechnological origin and also mixtures thereof. The orally effective hypoglycaemic active compounds preferably include sulphonylureas, biguanides, meglitinide derivatives, glucosidase inhibitors and PPAR-gamma agonists.

In a preferred embodiment of- the invention, the compounds according to the invention are administered in combination with insulin.

In a preferred embodiment of the invention, the compounds according to the invention are administered in combination with a sulphonylurea, such as, by way of example and by way of preference, tolbutamide, glibenclamide, glimepiride, glipizide or gliclazide.

In a preferred embodiment of the invention, the compounds according to the invention are administered in combination with a biguanide, such as, by way of example and by way of preference, metformin.

In a preferred embodiment of the invention, the compounds according to the invention are administered in combination with a meglitinide derivative, such as, by way of example and by way of preference, repaglinide or nateglinide.

In a preferred embodiment of the invention, the compounds according to the invention are administered in combination with a glucosidase inhibitor, such as, by way of example and by way of preference, miglitol or acarbose.

In a preferred embodiment of the invention, the compounds according to the invention are administered in combination with a PPAR-gamma agonist, for example from the class of the thiazolidinediones, such as, by way of example and by way of preference, pioglitazone or rosiglitazone.

The hypertensive agents are preferably understood as meaning compounds from the group of the calcium antagonists, angiotensin AII antagonists, ACE inhibitors, beta-receptor blockers, alpha-receptor blockers and diuretics.

In a preferred embodiment of the invention, the compounds according to the invention are administered in combination with a calcium antagonist, such as, by way of example and by way of preference, nifedipine, amlodipine, verapamil or diltiazem.

In a preferred embodiment of the invention, the compounds according to the invention are administered in combination with an angiotensin AII antagonist, such as, by way of example and by way of preference, losartan, valsartan, candesartan, embusartan or telmisartan.

In a preferred embodiment of the invention, the compounds according to the invention are administered in combination with an ACE inhibitor, such as, by way of example and by way of preference, enalapril, captopril, ramipril, delapril, fosinopril, quinopril, perindopril or trandopril.

In a preferred embodiment of the invention, the compounds according to the invention are administered in combination with a beta-receptor blocker, such as, by way of example and by way of preference, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol oder bucindolol.

In a preferred embodiment of the invention, the compounds according to the invention are administered in combination with an alpha-receptor blocker, such as, by way of example and by way of preference, prazosin.

In a preferred embodiment of the invention, the compounds according to the invention are administered in combination with a diuretic, such as, by way of example and by way of preference, furosemide.

In a preferred embodiment of the invention, the compounds according to the invention are administered in combination with antisympathotonics, such as reserpine, clonidine or alpha-methyldopa, with potassium channel-agonists, such as minoxidil, diazoxide, dihydralazine or hydralazine, or with nitrous oxide-releasing compounds, such as glycerol nitrate or sodium nitroprusside.

Antithrombotics are to be understood as meaning, preferably, compounds from the group of the platelet aggregation inhibitors or the anticoagulants.

In a preferred embodiment of the invention, the compounds according to the invention are administered in combination with a platelet aggregation inhibitor, such as, by way of example and by way of preference, aspirin, clopidogrel, ticlopidine or dipyridamol.

In a preferred embodiment of the invention, the compounds according to the invention are administered in combination with a thrombin inhibitor, such as, by way of example and by way of preference, ximelagatran, melagatran, bivalirudin or clexane.

In a preferred embodiment of the invention, the compounds according to the invention are administered in combination with a GPIIb/IIIa antagonist, such as, by way of example and by way of preference, tirofiban or abciximab.

In a preferred embodiment of the invention, the compounds according to the invention are administered in combination with a factor Xa inhibitor, such as, by way of example and by way of preference, DX-9065a, DPC 906, JTV 803, BAY 59-7939, DU-176b, fidexaban, razaxaban, fondaparinux, idraparinux, PMD-3112, YM-150, KFA-1982, EMD-503982, MCM-17, MLN-1021, SSR-126512 or SSR-128428.

In a preferred embodiment of the invention, the compounds according to the invention are administered in combination with heparin or a low molecular weight (LMW) heparin derivative.

In a preferred embodiment of the invention, the compounds according to the invention are administered in combination with a vitamin K antagonist, such as, by way of example and by way of preference, coumarin.

The present invention furthermore provides medicaments comprising at least one compound according to the invention, usually together with one or more inert non-toxic pharmaceutically suitable auxiliaries, and their use for the purposes mentioned above.

The compounds according to the invention can act systemically and/or locally. For this purpose, they can be administered in a suitable manner, such as, for example, orally, parenterally, pulmonally, nasally, sublingually, lingually, buccally, rectally, dermally, transdermally, conjunctivally, otically or as an implant or stent.

For these administration routes, the compounds according to the invention can be administered in suitable administration forms.

Suitable for oral administration are administration forms which work in accordance with the prior art and release the compounds according to the invention rapidly and/or in modified form and which comprise the compounds according to the invention in crystalline and/or amorphicized and/or dissolved form, such as, for example, tablets (uncoated or coated tablets, for example with enteric coats or coats which dissolve in a delayed manner or are insoluble and which control the release of the compounds according to the invention), films/wafers or tablets which dissolve rapidly in the oral cavity, films/lyophilizates, capsules (for example hard or soft gelatin capsules), sugar-coated tablets, granules, pellets, powders, emulsions, suspensions, aerosols or solutions.

Parenteral administration may take place by circumventing a bioabsorption step (for example intravenously, intraarterially, intracardially, intraspinally or intralumbarly), or with bioabsorption (for example intramuscularly, subcutaneously, intracutaneously, percutaneously or intraperitoneally). Administration forms suitable for parenteral administration are inter alia preparations for injection or infusion in the form of solutions, suspensions, emulsions, lyophilizates or sterile powders.

Suitable for other administration routes are, for example; medicaments suitable for inhalation (inter alia powder inhalers, nebulizers), nose drops, solutions or sprays, tablets to be administered lingually, sublingually or buccally, films/wafers or capsules, suppositories, preparations to be administered to ears or eyes, vaginal capsules, aqueous suspensions (lotions, shaking mixtures), lipophilic suspensions, ointments, creams, transdermal therapeutic systems (for example plasters), milk, pastes, foams, powders for pouring, implants or stents.

Preference is given to oral or parenteral administration, in particular to oral administration.

The compounds according to the invention can be converted into the administration forms mentioned. This can be carried out in a manner known per se by mixing with inert non-toxic pharmaceutically suitable auxiliaries. These auxiliaries include inter alia carriers (for example microcrystalline cellulose, lactose, mannitol), solvents (for example liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides), and flavour and/or odour corrigents.

In general, it has been found to be advantageous in the case of parenteral administration to administer amounts of about 0.001 to 1 mg/kg, preferably about 0.01 to 0.5 mg/kg, of body weight to obtain effective results. In the case of oral administration, the dosage is from about 0.01 to 100 mg/kg, preferably from about 0.01 to 20 mg/kg and very particularly preferably from 0.1 to 10 mg/kg of body weight.

In spite of this, it may be necessary to deviate from the amounts mentioned, namely depending on body weight, administration route, individual response to the active compound, the type of preparation and the time or the interval at which administration takes place. Thus, in some cases it may be sufficient to administer less than the abovementioned minimum amount, whereas in other cases the upper limit mentioned has to be exceeded. In the case of the administration of relatively large amounts, it may be expedient to divide these into a plurality of individual doses which are administered over the course of the day.

The working examples below illustrate the invention. The invention is not limited to the examples.

The percentages in the tests and examples below are, unless indicated otherwise, percentages by weight; parts are parts by weight. Solvent ratios, dilution ratios and concentrations of liquid/liquid solutions are in each case based on volume.

### A. EXAMPLES

### ABBREVIATIONS AND ACRONYMS

aq. aqueous
TLC thin-layer chromatography
DCI direct chemical ionization (in MS)
DMF dimethylformamide
DMSO dimethyl sulphoxide
eq. equivalent(s)
ESI electrospray ionization (in MS)
Et ethyl
GC gas chromatography
h hour(s)
HPLC high-pressure, high-performance liquid chromatography
LC-MS liquid chromatography-coupled mass spectroscopy
min minute(s)
MS mass spectroscopy
NMR nuclear magnetic resonance spectroscopy
Ph phenyl
RP reverse phase (in HPLC)
RT room temperature
Rₜ retention time (in HPLC)
THF tetrahydrofuran
UV ultraviolet spectroscopy

LC-MS and HPLC methods:

Method 1:

MS instrument type: Micromass ZQ; HPLC instrument type: Waters Alliance 2795; column: Phenomenex Synergi 2 μ Hydro-RP Mercury 20 mm×4 mm; mobile phase A: 1 l of water +0.5 ml of 50% strength formic acid, mobile phase B: 1 l of acetonitrile +0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 2:

MS instrument type: Micromass ZQ; HPLC instrument type: HP 1100 Series; UV DAD; column: Phenomenex Synergi 2 μ Hydro-RP Mercury 20 mm×4 mm; mobile phase A: 1 l of water +0.5 ml of 50% strength formic acid, mobile phase B: 1 l of acetonitrile +0.5 ml of 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 3:

Instrument: Micromass Quattro LCZ with HPLC Agilent Series 1100; column: Phenomenex Synergi 2 μ Hydro-RP Mercury 20 mm×4 mm; mobile phase A: 1 l of water +0.5 ml of 50% strength formic acid, mobile phase B: 1 l of acetonitrile +0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

Method 4:

Instrument: Micromass Platform LCZ with HPLC Agilent Series 1100; column: Phenomenex Synergi 2 μ Hydro-RP Mercury 20 mm×4 mm; mobile phase A: 1 l of water +0.5 ml of 50% strength formic acid, mobile phase B: 1 l of acetonitrile +0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 5:

Instrument: Micromass Platform LCZ with HPLC Agilent Series 1100; column: Thermo HyPURITY Aquastar 3 μ 50 mm×2.1 mm; mobile phase A: 1 l of water +0.5 ml of 50% strength formic acid, mobile phase B: 1 l of acetonitrile +0.5 ml of 50% strength formic acid; gradient: 0.0min 100% A→0.2 min 100% A→2.9 min 30% A→3.1 min 10% A→5.5 min 10% A; oven: 50° C.; flow rate: 0.8 ml/min; UV detection: 210 nm.

Method 6:

MS instrument type: Micromass ZQ; HPLC instrument type: Waters Alliance 2795; column: Merck Chromolith SpeedROD RP-18e 50 mm×4.6 mm; mobile phase A: 1 l of water +0.5 ml of 50% strength formic acid; mobile phase B: 1 l of acetonitrile +0.5 ml of 50% strength formic acid; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

Method 7:

Instrument: HP 1100 with DAD detection; column: Kromasil 100 RP-18, 60 mm×2.1 mm, 3.5 μm; mobile phase A: 5 ml of HClO₄ (70% strength)/l of water, mobile phase B: acetonitrile; gradient: 0 min 2% B→0.5 min 2% B→4.5 min 90% B→9 min 90% B→9.2 min 2% B→10 min 2% B; flow rate: 0.75 ml/min; column temperature: 30° C.; UV detection: 210 nm.

Method 8:

Instrument: HP 1100 with DAD detection; column: Kromasil 100 RP-18, 60 mm×2.1 mm, 3.5 μm; mobile phase A: 5 ml of HClO₄ (70% strength)/l of water, mobile phase B: acetonitrile; gradient: 0 min 2% B→0.5 min 2% B→4.5 min 90% B→15 min 90% B→15.2 min 2% B→16 min 2% B; flow rate: 0.75 ml/min; column temperature: 30° C.; UV detection: 210 nm.

Method 9:

Instrument: HP 1100 with DAD detection; column: Kromasil 100 RP-18, 60 mm×2.1 mm, 3.5 μm; mobile phase A: 5 ml of HClO₄ (70% strength)/l of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

Method 10:

MS instrument: Micromass TOF (LCT); HPLC instrument: 2-column setup, Waters 2690; column: YMC-ODS-AQ, 50 mm×4.6 mm, 3.0 μm; mobile phase A: water +0.1% formic acid, mobile phase B: acetonitrile +0.1% formic acid; gradient: 0.0 min 100% A→0.2 min 95% A→1.8 min 25% A→1.9 min 10% A→2.0 min 5% A→3.2 min 5% A; oven: 40° C.; flow rate: 3.0 ml/min; UV detection: 210 nm.

Method 11:

Instrument: Micromass Platform LCZ with HPLC Agilent Series 1100; column: Thermo Hypersil GOLD 3 μ, 20 mm×4 mm; mobile phase A: 1 l of water +0.5 ml of 50% strength formic acid, mobile phase B: 1 l of acetonitrile +0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Starting Materials and Intermediates:

### Example 1A

### Ethyl 4-hydroxy-2-phenylpyrimidine-5-carboxylate

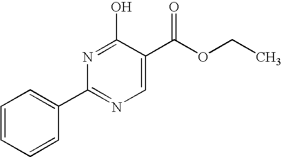

10.0 g of benzamidine hydrochloride (63.9 mmol) and a solution of 13.8 g of diethyl 2-ethoxymethylenemalonate (63.9 mmol) in 25 ml of ethanol are added to 47.7 ml of ethanolic sodium ethoxide solution (21%, 41.40 g, 128 mmol). The mixture is heated under reflux for 2 h and then poured into 100 ml of 6 N hydrochloric acid. The precipitated solid is filtered off with suction, washed with water and dried. This gives 11.6 g (73% of theory) of the product.

¹H-NMR (400 MHz, DMSO-d₆): δ=1.30 (t, 3H), 4.27 (q, 2H), 7.52-7.59 (m, 2H), 7.61-7.68 (m, 1H), 8.17 (d, 2H), 8.66 (s, 1H), 13.17 (br. s, 1H).

LC-MS (Method 3): Rₜ=1.65 min; m/z=245.1 [M+H]⁺.

### Example 2A

### Ethyl 4-chloro-2-phenylpyrimidine-5-carboxylate

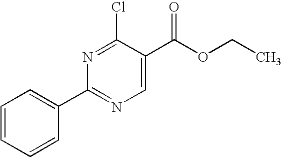

At room temperature, 6.57 ml of thionyl chloride (10.70 g, 90.1 mmol) are slowly added dropwise to 11.00 g of the compound from Example 1A (45.0 mmol) in 120 ml of DMF. The mixture is stirred at room temperature for 2 h. 7.50 g of potassium carbonate (54.0 mmol) are then added, and the mixture is poured into 100 ml of ice-water. The precipitated solid is filtered off with suction, washed with water and dried in a vacuum drying cabinet at 30° C. This gives 11.4 g (96% of theory) of the product.

¹H-NMR (400 MHz, DMSO-d₆): Δ=1.38 (t, 3H), 4.40 (q, 2H), 7.56-7.68 (m, 3H), 8.40 (d, 2H), 9.26 (s, 1H).

HPLC (Method 9): Rₜ=5.20 min.

MS (DCI): m/z=263 [M+H]⁺.

### Example 3A

### 2-Fluorobenzenecarboximidamide hydrochloride

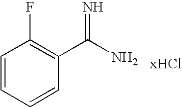

Under argon, 2.65 g of ammonium chloride (49.5 mmol) are suspended in 70 ml of toluene. At 0° C., 3.57 g of trimethylaluminium (49.5 mmol) are added slowly. The mixture is stirred at room temperature until the evolution of gas has ceased. 3.00 g of 2-fluorobenzonitrile (24.8 mmol) are then added, and the mixture is heated under reflux overnight. After cooling to room temperature, 10 g of silica gel are added and the mixture is stirred for 15 min. The silica gel is filtered off and washed with methanol/methylene chloride (1:1). The filtrate is concentrated under reduced pressure and the residue is washed with methylene chloride/methanol (10:1) and then with methylene chloride. The residue obtained is 2.50 g (58% of theory) of the product.

¹H-NMR (300 MHz, DMSO-d₆): δ=7.39-7.51 (m, 2H), 7.65-7.77 (m, 2H), 9.45 (s, 4H).

HPLC (Method 9): Rₜ=0.99 min.

MS (DCI): m/z=139.1 [M+H]⁺.

### Example 4A

### Ethyl 2-(3-fluoro-4-methylphenyl)-4-hydroxypyrimidine-5-carboxylate

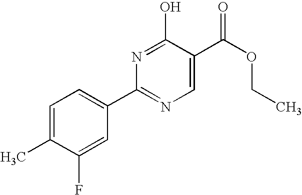

The title compound is prepared by a reaction sequence analogous to the one described in Example 1A.

LC-MS (Method 3): Rₜ=1.99 min; m/z=277.2 [M+H]⁺.

¹H-NMR (400 MHz, DMSO-d₆): δ=1.28 (t, 3H), 2.32 (s, 3H), 4.24 (q, 2H), 7.47 (dd, 1H), 7.91-7.99 (m, 2H), 8.61 (s, 1H).

### Example 5A

### Ethyl 4-chloro-2-(3-fluoro-4-methylphenyl)pyrimidine-5-carboxylate

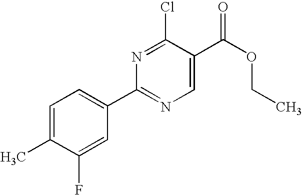

The title compound is prepared starting from Example 4A by a reaction sequence analogous to the one described in Example 2A.

LC-MS (Method 3): Rₜ=3.04 min; m/z=295.1 [M+H]⁺.

¹H-NMR (300 MHz, DMSO-d₆): δ=1.36 (t, 3H), 2.34 (s, 3H), 4.39 (q, 2H), 7.52 (dd, 1H), 8.03 (dd, 1H), 8.15 (dd, 1H), 9.26 (s, 1H).

### Example 6A

### Ethyl 2-[3,5-di(trifluoromethyl)phenyl]-4-hydroxypyrimidine-5-carboxylate

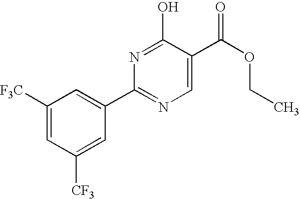

The title compound is prepared by a reaction sequence analogous to the one described in Example 1A.

LC-MS (Method 3): Rₜ=2.61 min; m/z=381.2 [M+H]⁺.

¹H-NMR (400 MHz, DMSO-d₆): δ=1.29 (t, 3H), 4.27 (q, 2H), 8.38 (s, 1H), 8.71 (s, 1H), 8.82 (s, 2H).

### Example 7A

### Ethyl 2-[3,5-di(trifluoromethyl)phenyl]-4-chloropyrimidine-5-carboxylate

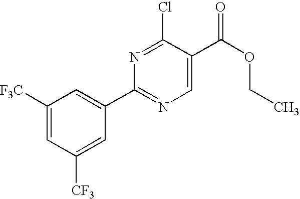

The title compound is prepared starting from Example 6A by a reaction sequence analogous to the one described in Example 2A.

LC-MS (Method 3): Rₜ=3.19 min; m/z=399.1 [M+H]⁺.

¹H-NMR (300 MHz, DMSO-d₆): δ=1.37 (t, 3H), 4.42 (q, 2H), 8.47 (s, 1H), 8.88 (s, 2H), 9.37 (s, 1H).

### Example 8A

### 3-Fluoro-4-(trifluoromethyl)benzenecarboximidamide hydrochloride

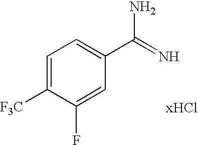

The title compound is prepared by a reaction sequence analogous to the one described in Example 3A.

HPLC (Method 9): Rₜ=3.66 min.

MS (DCI): m/z=206.9 [M+H]⁺.

¹H-NMR (300 MHz, DMSO-d₆): δ=7.85 (d, 1H), 7.99-8.14 (m, 2H), 9.50 (br. s, 4H).

### Example 9A

### Ethyl 2-[3-fluoro-4-(trifluoromethyl)phenyl]-4-hydroxypyrimidine-5-carboxylate

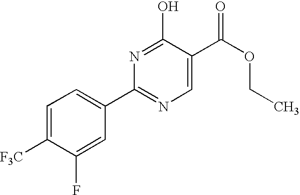

The title compound is prepared starting from Example 8A by a reaction sequence analogous to the one described in Example 1A.

LC-MS (Method 3): Rₜ=2.32 min; m/z=331.3 [M+H]⁺.

¹H-NMR (300 MHz, DMSO-d₆): δ=1.30 (t, 3H), 4.28 (q, 2H), 7.97-8.06 (m, 1H), 8.18-8.28 (m, 2H), 8.72 (s, 1H).

### Example 10A

### Ethyl 4-chloro-2-[3-fluoro-4-(trifluoromethyl)phenyl]pyrimidine-5-carboxylate

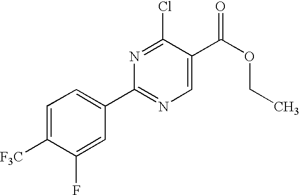

The title compound is prepared starting from Example 9A by a reaction sequence analogous to the one described in Example 2A.

LC-MS (Method 3): Rₜ=3.17 min; m/z=349.2 [M+H]⁺.

¹H-NMR (400 MHz, DMSO-d₆): δ=1.37 (t, 3H), 4.41 (q, 2H), 8.01-8.07 (m, 1H), 8.32 (d, 1H), 8.40 (d, 1H), 9.34 (s, 1H).

### Example 11A

### 4-Chloro-3-methylbenzenecarboximidamide hydrochloride

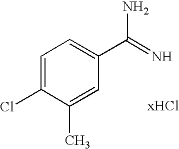

The title compound is prepared by a reaction sequence analogous to the one described in Example 3A.

HPLC (Method 9): Rₜ=3.35 min.

MS (DCI): m/z=169.0 [M+H]⁺.

¹H-NMR (300 MHz, DMSO-d₆): δ=2.43 (s, 3H), 7.61 (d, 1H), 7.74 (dd, 1H), 7.95 (d, 1H), 9.31 (br. s, 4H).

### Example 12A

### Ethyl 2-(4-chloro-3-methylphenyl)-4-hydroxypyrimidine-5-carboxylate

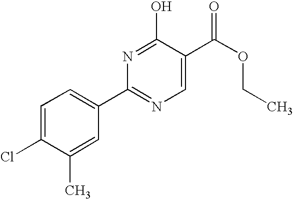

The title compound is prepared starting from Example 11A by a reaction sequence analogous to the one described in Example 1A.

¹H-NMR (300 MHz, DMSO-d₆): δ=1.29 (t, 3H), 2.41 (s, 3H), 4.26 (q, 2H), 7.55 (d, 1H), 8.06 (dd, 1H), 8.23 (d, 1H), 8.64 (s, 1H).

### Example 13A

### Ethyl 4-chloro-2-(4-chloro-3-methylphenyl)pyrimidine-5-carboxylate

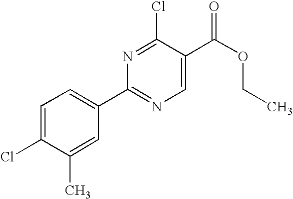

The title compound is prepared starting from Example 12A by a reaction sequence analogous to the one described in Example 2A.

LC-MS (Method 3): Rₜ=3.28 min; m/z=311.2 [M+H]⁺.

¹H-NMR (400 MHz, DMSO-d₆): δ=1.36 (t, 3H), 2.43 (s, 3H), 4.39 (q, 2H), 7.59 (d, 1H), 8.25 (dd, 1H), 8.33 (d, 1H), 9.26 (s, 1H).

### Example 14A

### 3,4-Dimethylbenzenecarboximidamide hydrochloride

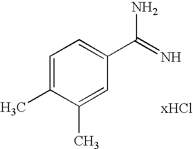

The title compound is prepared by a reaction sequence analogous to the one described in Example 3A.

HPLC (Method 9): Rₜ=3.43 min.

MS (DCI): m/z=149.0 [M+H]⁺.

¹H-NMR (300 MHz, DMSO-d₆): δ=2.30 (s, 3H), 2.32 (s, 3H), 7.38 (d, 1H), 7.59 (dd, 1H), 7.66 (d, 1H), 9.20 (br. s, 4H).

### Example 15A

### Ethyl 2-(3,4-dimethylphenyl)-4-hydroxypyrimidine-5-carboxylate

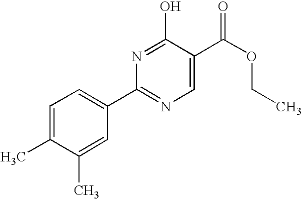

The title compound is prepared starting from Example 14A by a reaction sequence analogous to the one described in Example 1A.

LC-MS (Method 3): Rₜ=1.96 min; m/z=273.3 [M+H]⁺.

¹H-NMR (300 MHz, DMSO-d₆): δ=1.28 (t, 3H), 2.30 (s, 6H), 4.25 (q, 2H), 7.31 (d, 1H), 7.92 (dd, 1H), 8.00 (s, 1H), 8.61 (s, 1H).

### Example 16A

### Ethyl 4-chloro-2-(3,4-dimethylphenyl)pyrimidine-5-carboxylate

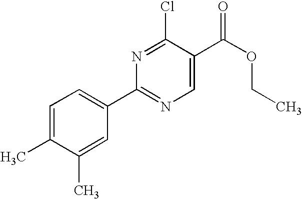

The title compound is prepared starting from Example 15A by a reaction sequence analogous to the one described in Example 2A.

LC-MS (Method 1): Rₜ=2.97 min; m/z=291.1 [M+H]⁺.

¹H-NMR (400 MHz, DMSO-d₆): δ=1.36 (t, 3H), 2.32 (s, 3H), 2.33 (s, 3H), 4.39 (q, 2H), 7.35 (d, 1H), 8.13 (d, 1H), 8.18 (s, 1H), 9.22 (s, 1H).

### Example 17A

### Ethyl 4-hydroxy-2-(2-fluorophenyl)pyrimidine-5-carboxylate

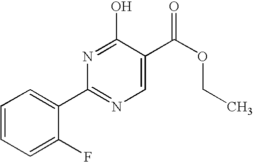

The title compound is prepared starting from Example 3A by a reaction sequence analogous to the one described in Example 1A.

¹H-NMR (400 MHz, DMSO-d₆): δ=1.30 (t, 3H), 4.26 (q, 2H), 7.35-7.46 (m, 2H), 7.62-7.71 (m, 1H), 7.73-7.82 (m, 1H), 8.61 (s, 1H), 13.30 (br. s, 1H).

LC-MS (Method 1): Rₜ=1.38 min; m/z=263.2 [M+H]⁺.

### Example 18A

### Ethyl 4-chloro-2-(2-fluorophenyl)pyrimidine-5-carboxylate

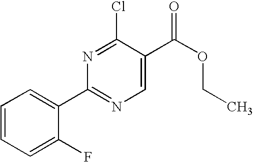

The title compound is prepared starting from Example 17A by a reaction sequence analogous to the one described in Example 2A.

1H-NMR (400 MHz, DMSO-d₆): δ=1.37 (t, 3H), 4.40 (q, 2H), 7.38-7.44 (m, 2H), 7.63-7.71 (m, 1H), 8.12 (dd, 1H), 9.31 (s, 1H).

HPLC (Method 7): Rₜ=4.60 min.

MS (ESIpos): m/z=280.2 [M+H]⁺.

### Example 19A

### Ethyl 4-hydroxy-2-(4-methyl-3-nitrophenyl)pyrimidine-5-carboxylate

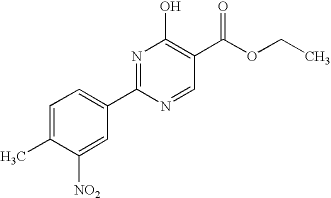

The title compound is prepared by a reaction sequence analogous to the one described in Example 1A.

LC-MS (Method 2): Rₜ=2.08 min; m/z=304.1 [M+H]⁺.

### Example 20A

### Ethyl 4-chloro-2-(4-methyl-3-nitrophenyl)pyrimidine-5-carboxylate

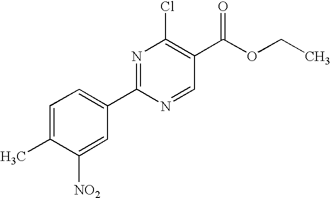

The title compound is prepared starting from Example 19A by a reaction sequence analogous to the one described in Example 2A.

LC-MS (Method 2): Rₜ=2.96 min; m/z=322.0 [M+H]⁺.

### Example 21A

### 4-Fluoro-3-methoxybenzenecarboximidamide hydrochloride

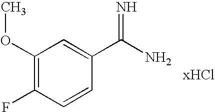

The title compound is prepared by a reaction sequence analogous to the one described in Example 3A.

LC-MS (Method 11): Rₜ=1.70 min; m/z=169.0 [M+H]⁺.

¹H-NMR (400 MHz, DMSO-d₆): δ=4.36 (s, 3H), 7.85-7.94 (m, 2H), 8.10 (d, 1H), 7.90 (br. s, 4H).

### Example 22A

### Ethyl 4-hydroxy-2-(4-fluoro-3-methoxyphenyl)pyrimidine-5-carboxylate

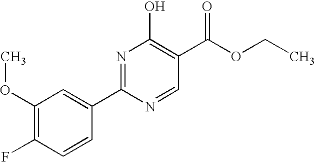

The title compound is prepared by a reaction sequence analogous to the one described in Example 1A.

LC-MS (Method 1): Rₜ=1.63 min; m/z=293.2 [M+H]⁺.

### Example 23A

### Ethyl 4-chloro-2-(4-fluoro-3-methoxyphenyl)pyrimidine-5-carboxylate

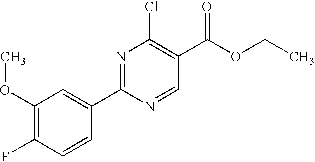

The title compound is prepared starting from Example 22A by a reaction sequence analogous to the one described in Example 2A.

LC-MS (Method 3): Rₜ=2.83 min; m/z=311.2 [M+H]⁺.

### Example 24A

### 3,4,5-Trifluorobenzenecarboximidamide hydrochloride

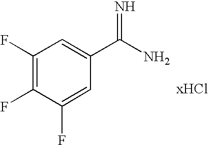

The title compound is prepared by a reaction sequence analogous to the one described in Example 3A.

LC-MS (Method 11): Rₜ=0.7 min; m/z=175.0 [M+H]⁺.

¹H-NMR (400 MHz, DMSO-d₆): δ=8.28-8.38 (m, 2H), 9.46 (br. s, 4H).

### Example 25A

### Ethyl 4-hydroxy-2-(3,4,5-trifluorophenyl)pyrimidine-5-carboxylate

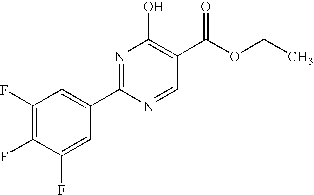

The title compound is prepared by a reaction sequence analogous to the one described in Example 1A.

LC-MS (Method 3): Rₜ=2.17 min; m/z=299.2 [M+H]⁺.

### Example 26A

### Ethyl 4-chloro-2-(3,4,5-trifluorophenyl)pyrimidine-5-carboxylate

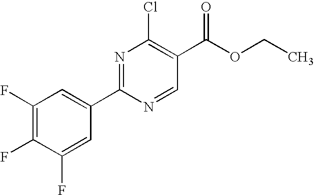

The title compound is prepared starting from Example 25A by a reaction sequence analogous to the one described in Example 2A.

LC-MS (Method 1): Rₜ=2.91 min; m/z=317.1 [M+H]⁺.

### Example 27A

### 3,4-Difluorobenzenecarboximidamide hydrochloride

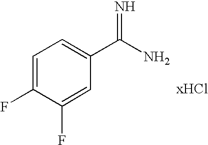

The title compound is prepared by a reaction sequence analogous to the one described in Example 3A.

LC-MS (Method 11): Rₜ=0.88 min; m/z=157.0 [M+H]⁺.

### Example 28A

### Ethyl 4-hydroxy-2-(3,4-difluorophenyl)pyrimidine-5-carboxylate

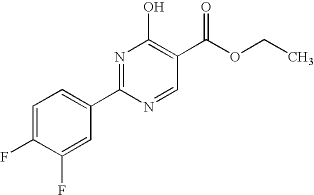

The title compound is prepared by a reaction sequence analogous to the one described in Example 1A.

LC-MS (Method 3): Rₜ=1.91 min; m/z=281.2 [M+H]⁺.

### Example 29A

### Ethyl 4-chloro-2-(3,4-difluorophenyl)pyrimidine-5-carboxylate

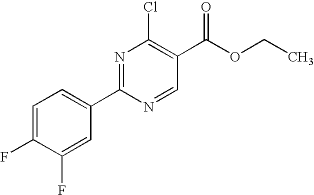

The title compound is prepared starting from Example 28A by a reaction sequence analogous to the one described in Example 2A.

LC-MS (Method 3): Rₜ=2.98 min; m/z=299.2 [M+H]⁺.

### Example 30A

### (2-Chlorobenzyl)zinc bromide

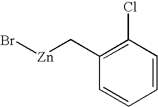

725.56 mg (11.1 mmol) of zinc dust in 2.5 ml of DMF and 84.11 mg (0.4 mmol) of 1,2-dibromethane are stirred at 70° C. for 10 minutes. After cooling to room temperature, 44.47 μl (0.4 mmol) of chlorotrimethylsilane are added and stirring is continued for a further 30 minutes. The mixture is then cooled to 0° C., and 2.00 g (9.7 mmol) of 2-chlorobenzyl bromide, dissolved in 10 ml of DMF, are added dropwise. After one hour at room temperature, the mixture is stirred for a further hour at 70° C. After cooling, 7.5 ml of DMF are added. The about 0.5 M solution of (2-chlorobenzyl)zinc bromide in DMF obtained in this manner is used as such for the next reaction (see Example 51).

### Working Examples

General Method 1 for preparing the phenoxy ester derivatives:

Sodium hydride (2.0 eq.) is added the phenol derivative (1.5 eq.) in acetonitrile, and the mixture is then stirred at room temperature for 10 minutes. The 2-chloropyrimidine derivative (1.0 eq.) is then added. The mixture is stirred at room temperature overnight and then concentrated, and water is added to the residue. The mixture is extracted twice with ethyl acetate. The aqueous phase is acidified with 1 N hydrochloric acid and extracted with ethyl acetate. The combined organic phases are dried over magnesium sulphate, and the solvent is removed under reduced pressure. The crude product is purified by column chromatography.

General method 2 for preparing the phenoxy ester derivatives:

Potassium carbonate (2.0 eq) is added to the phenol derivative (1.2 eq.) and the 2-chloropyrimidine derivative (1.0 eq.) in N,N-dimethylformamide, and the mixture is then stirred at room temperature overnight. The mixture is filtered off with suction and the residue is washed with a little THF. The filtrate is concentrated. The crude product is purified by column chromatography.

General method 3 for preparing the phenoxycarboxylic acid derivatives (Examples 34-38):

The compound from Example 2A (100 μm) and the phenol derivative (100 μm) in DMF (500 μl) are combined, potassium carbonate (2 eq.) is then added and the mixture is stirred at room temperature overnight. 0.2 ml of ethanol and 0.2 ml of 1 N aqueous sodium hydroxide solution are then added, and the mixture is stirred at room temperature for 2 h. After addition of 0.1 ml of 2 N hydrochloric acid and dilution with DMSO, the mixture is directly purified by chromatography.

### Example 1

### Ethyl 4-(2-chlorophenoxy)-2-phenylpyrimidine-5-carboxylate

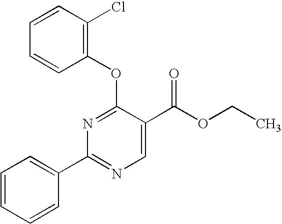

76 mg of sodium hydride (1.9 mmol) are added to 183 mg of 2-chlorophenol (1.4 mmol) in 3 ml of acetonitrile, and the mixture is stirred at room temperature for 10 min. 250 mg of the compound from Example 2A (0.9 mmol) are then added. The mixture is stirred at room temperature overnight and then poured into 20 ml of water. The mixture is extracted twice with in each case 20 ml of methylene chloride. The combined organic phases are washed with 20 ml of I N aqueous sodium hydroxide solution and dried over magnesium sulphate, and the solvent is removed under reduced pressure. This gives 335 mg (99% of theory) of the product.

¹H-NMR (300 MHz, DMSO-d₆): δ=1.38 (t, 3H), 4.41 (q, 2H), 7.39-7.59 (m, 5H), 7.70 (d, 1H), 8.01-8.07 (m, 2H), 9.24 (s, 1H).

LC-MS (Method 2): Rₜ=3.13 min; m/z=355.2 [M+H]⁺.

### Example 2

### 4-(2-Chlorophenoxy)-2-phenylpyrimidine-5-carboxylic acid

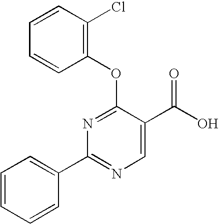

37 mg of sodium hydroxide (0.9 mmol) are added to 330 mg of the compound from Example 1 (0.9 mmol) in 1.50 ml of dioxane. The mixture is stirred at room temperature overnight and then added to 10 ml of water. The mixture is acidified with 1 N hydrochloric acid and then extracted three times with in each case 10 ml of methylene chloride. The combined organic phases are dried over magnesium sulphate, and the solvent is removed under reduced pressure. The residue is purified by preparative HPLC (mobile phase: acetonitrile/water with 0.1% of formic acid, gradient 10:90→95:5). This gives 262 mg (86% of theory) of the product.

¹H-NMR (300 MHz, DMSO-d₆): δ=7.38-7.59 (m, 6H), 7.70 (dd, 1H), 8.03 (dd, 2H), 9.22 (s, 1H), about 13.60 (br. s, 1H).

LC-MS (Method 3): Rₜ=2.43 min; m/z=327.2 [M+H]⁺.

### Example 3

### 4-(2-Fluorophenoxy)-2-phenylpyrimidine-5-carboxylic acid

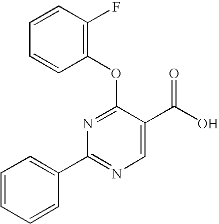

The title compound is prepared analogously to Examples 1 and 2.

¹H-NMR (400 MHz, DMSO-d₆): δ=7.32-7.56 (m, 7H), 8.06 (d, 2H), 9.18 (s, 1H).

HPLC (Method 9): Rₜ=4.53 min.

MS (ESIpos): m/z=311.2 [M+H]⁺.

### Example 4

### 4-(2-Methylphenoxy)-2-phenylpyrimidine-5-carboxylic acid

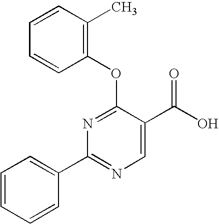

The title compound is prepared analogously to Examples 1 and 2.

¹H-NMR (400 MHz, DMSO-d₆): δ=2.12 (s, 3H), 7.22-7.30 (m, 2H), 7.30-7.37 (m, 1H), 7.38-7.48 (m, 3H), 7.49-7.54 (m, 1H), 8.05 (d, 2H), 9.16 (s, 1H), 13.53 (br. s, 1H).

HPLC (Method 9): Rₜ=4.67 min.

MS (ESIpos): m/z=307.3 [M+H]⁺.

### Example 5

### 4-(2-Bromophenoxy)-2-phenylpyrimidine-5-carboxylic acid

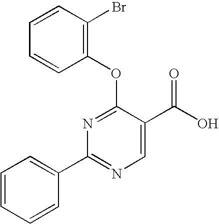

The title compound is prepared analogously to Examples 1 and 2.

¹H-NMR (400 MHz, DMSO-d₆): δ=7.31-7.38 (m, 1H), 7.42-7.59 (m, 5H), 7.83 (d, 1H), 8.03 (d, 2H), 9.22 (s, 1H), about 13.50 (br. s, 1H).

LC-MS (Method 3): Rₜ=2.46 min; m/z=371.1 [M+H]⁺.

### Example 6

4-(2-Chloro-4-methylphenoxy)-2-phenylpyrimidine-5-carboxylic acid

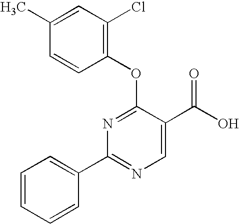

The title compound is prepared analogously to Examples 1 and 2.

¹H-NMR (400 MHz, DMSO-d₆): δ=7.36-7.56 (m, 8H), 7.76 (d, 2H), 7.83 (d, 2H), 8.14 (d, 2H), 9.19 (s, 1H), about 13.60 (br. s, 1H).

HPLC (Method 7): Rₜ=5.09 min.

MS (ESIpos): m/z=369.4 [M+H]⁺.

### Example 7

### 4-(2-Chloro-4-methoxyphenoxy)-2-phenylpyrimidine-5-carboxylic acid

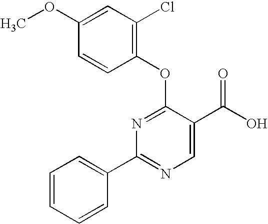

The title compound is prepared analogously to Examples 1 and 2.

¹H-NMR (400 MHz, DMSO-d₆): δ=3.85 (s, 3H), 7.07 (dd, 1H), 7.27 (d, 1H), 7.41 (d, 1H), 7.44-7.57 (m, 3H), 8.06 (d, 2H), 9.30 (s, 1H), 13.61 (br. s, 1H).

HPLC (Method 9): Rₜ=4.67 min.

MS (ESIpos): m/z=357.0 [M+H]⁺.

### Example 8

### 4-(2,5-Dichlorophenoxy)-2-phenylpyrimidine-5-carboxylic acid

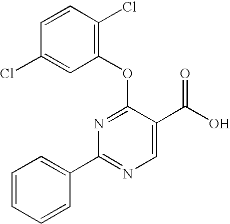

The title compound is prepared analogously to Examples 1 and 2.

¹H-NMR (400 MHz, DMSO-d₆): δ=7.45-7.58 (m, 4H), 7.72-7.78 (m, 2H), 8.06 (d, 2H), 9.24 (s, 1H).

HPLC (Method 7): Rₜ=4.89 min.

MS (ESIpos): m/z=360.9 [M+H]⁺.

### Example 9

### Ethyl 4-(2,5-dimethylphenoxy)-2-phenylpyrimidine-5-carboxylate

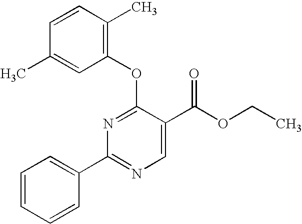

The title compound is prepared analogously to Example 1.

¹H-NMR (400 MHz, DMSO-d₆): δ=1.35 (t, 3H), 2.09 (s, 2H), 2.34 (s, 3H), 4.40 (q, 2H), 7.06-7.12 (m, 2H), 7.28 (d, 1H), 7.42-7.58 (m, 3H), 8.06 (d, 2H), 9.20 (s, 1H).

HPLC (Method 9): Rₜ=5.67 min.

MS (ESIpos): m/z=349.1 [M+H]⁺.

### Example 10

### 4-(2,5-Dimethylphenoxy)-2-phenylpyrimidine-5-carboxylic acid

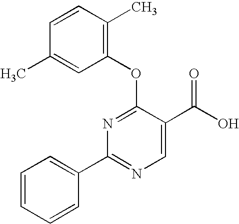

The title compound is prepared analogously to Example 2.

¹H-NMR (400 MHz, DMSO-d₆): δ=2.07 (s, 3H), 2.32 (s, 3H), 7.05 (s, 1H), 7.08 (d, 1H), 7.28 (d, 1H), 7.42-7.49 (m, 2H), 7.50-7.55 (m, 1H), 8.06 (d, 2H), 9.18 (s, 1H).

HPLC (Method 7): Rₜ=4.83 min.

MS (ESIpos): m/z=321.0 [M+H]⁺.

### Example 11

### 4-(2-Chlorophenoxy)-2-(3-fluorophenyl)pyrimidine-5-carboxylic acid

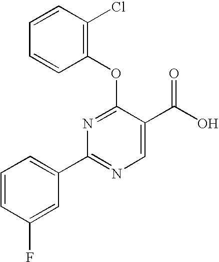

The title compound is prepared analogously to Examples 1 and 2.

¹H-NMR (300 MHz, DMSO-d₆): δ=7.37-7.58 (m, 5H), 7.63-7.73 (m, 2H), 7.87 (d, 1H), 9.22 (s, 1H), 13.71 (br. s, 1H).

LC-MS (Method 3): Rₜ=2.52 min; m/z=345.1 [M+H]⁺.

### Example 12

### 4-(2-Chlorophenoxy)-2-(4-methylphenyl)pyrimidine-5-carboxylic acid

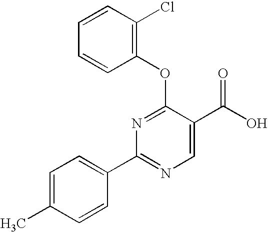

The title compound is prepared analogously to Examples 1 and 2.

¹H-NMR (300 MHz, DMSO-d₆): δ=2.32 (s, 3H), 7.27 (d, 2H), 7.39-7.45 (m, 1H), 7.45-7.57 (m, 2H), 7.70 (dd, 1H), 8.01 (d, 2H), 9.20 (s, 1H), 13.58 (br. s, 1H).

LC-MS (Method 3): Rₜ=2.58 min; m/z=341.2 [M+H]⁺.

### Example 13

### 4-(2-Chlorophenoxy)-2-(4-fluorophenyl)pyrimidine-5-carboxylic acid

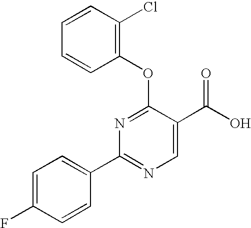

The title compound is prepared analogously to Examples 1 and 2.

¹H-NMR (300 MHz, DMSO-d₆): δ=7.27-7.36 (m, 2H), 7.39-7.46 (m, 1H), 7.46-7.55 (m, 2H), 7.70 (dd, 1H), 8.02-8.11 (m, 2H), 9.21 (s, 1H), about 13.62 (br. s, 1H).

HPLC (Method 7): Rₜ=4.73 min.

MS (DCI): m/z=345.1 [M+H]⁺.

### Example 14

### 4-(2-Chlorophenoxy)-2-(4-methoxyphenyl)pyrimidine-5-carboxylic acid

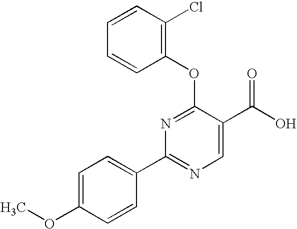

The title compound is prepared analogously to Examples 1 and 2.

¹H-NMR (400 MHz, DMSO-d₆): δ=3.30 (s, 3H), 7.00 (d, 2H), 7.39-7.44 (m, 1H), 7.45-7.54 (m, 2H), 7.79 (d, 1H), 7.98 (d, 2H), 9.16 (s, 1H), 13.50 (br. s, 1H).

HPLC (Method 7): Rₜ=4.60 min.

MS (ESIpos): m/z=357.2 [M+H]⁺.

### Example 15

### Ethyl 4-[2-chloro-5-(trifluoromethyl)phenoxy]-2-phenylpyrimidine-5-carboxylate

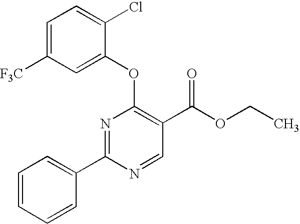

168.0 mg (0.9 mmol) of 2-chloro-5-(trifluoromethyl)phenol, 45.0 mg (1.1 mmol) of sodium hydride and 150.0 mg (0.6 mmol) of the compound from Example 2A are reacted according to the General Method 1.

Yield: 103 mg (43% of theory)

LC-MS (Method 1): Rₜ=3.09 min; m/z=423.1 [M+H]⁺.

¹H-NMR (400 MHz, DMSO-d₆): δ=1.36 (t, 3H), 4.41 (q, 2H), 7.44-7.51 (m, 2H), 7.52-7.58 (m, 1H), 7.80 (d, 1H), 7.93 (dd, 1H), 8.19 (d, 1H), 9.29 (s, 1H).

### Example 16

### Ethyl 4-(5-chloro-2-methylphenoxy)-2-phenylpyrimidine-5-carboxylate

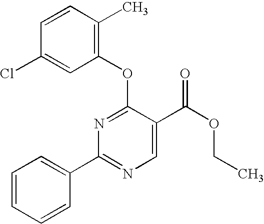

130.26 mg (0.9 mmol) of 5-chloro-2-methylphenol, 200.0 mg (0.8 mmol) of the compound from Example 2A and 210.44 mg (1.5 mmol) of potassium carbonate are reacted according to the General Method 2.

Yield: 232 mg (83% of theory)

LC-MS (Method 2): Rₜ=3.34 min; m/z=369.1 [M+H]⁺.

¹H-NMR (300 MHz, DMSO-d₆): δ=1.36 (t, 3H), 2.13 (s, 3H), 4.39 (q, 2H), 7.36 (dd, 1H), 7.43-7.58 (m, 5H), 8.05-8.11 (m, 2H), 9.21 (s, 1H).

### Example 17

### Ethyl 4-(2-chlorophenoxy)-2-(3-fluoro-4-methylphenyl)pyrimidine-5-carboxylate

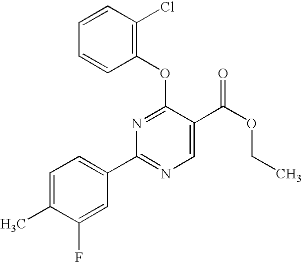

104.69 mg (0.1 mmol) of 2-chlorophenol, 200.0 mg (0.7 mmol) of the compound from Example 5A and 187.58 mg (1.4 mmol) of potassium carbonate are reacted according to the General Method 2.

Yield: 236 mg (90% of theory)

LC-MS (Method 3): Rₜ=3.18 min; m/z=387.1 [M+H]⁺.

¹H-NMR (300 MHz, DMSO-d₆): δ=1.36 (t, 3H), 2.26 (s, 3H), 4.40 (q, 2H), 7.36-7.47 (m, 2H), 7.49-7.56 (m, 2H), 7.63 (dd, 1H), 7.70 (dd, 1H), 7.78 (dd, 1H), 9.23 (s, 1H).

### Example 18

### 4-(2-Chlorophenoxy)-2-(3-fluoro-4-methylphenyl)pyrimidine-5-carboxylic acid

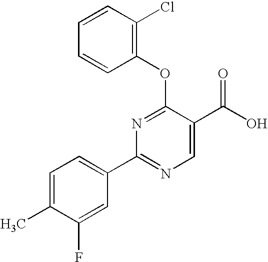

1.07 ml (1.1 mmol) of 1 N aqueous sodium hydroxide solution are added to 345.0 mg (0.9 mmol) of the compound from Example 17 in 6 ml of ethanol/tetrahydrofuran (1:2). The solution is stirred at room temperature overnight and then concentrated. The mixture is taken up in water and acidified with 1 N hydrochloric acid and the milky solution is then filtered off with suction. The residue is dissolved in 5 ml of ethyl acetate and washed with 5 ml of saturated sodium chloride solution. The phases are separated, the organic phase is then dried over magnesium sulphate and the solvent is removed under reduced pressure. This gives 311 mg (97% of theory) of the product.

LC-MS (Method 3): Rₜ=2.68 min; m/z=359.2 [M+H]⁺.

¹H-NMR (300 MHz, DMSO-d₆): δ=2.26 (s, 3H), 7.35-7.56 (m, 4H), 7.49-7.56 (m, 2H), 7.62 (dd, 1H), 7.70 (dd, 1H), 7.76 (dd, 1H), 9.21 (s, 1H).

### Example 19

### Ethyl 4-(2,5-dichlorophenoxy)-2-(3-fluoro-4-methylphenyl)pyrimidine-5-carboxylate

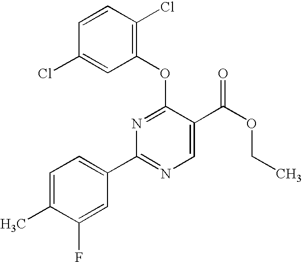

132.72 mg (0.8 mmol) of 2,5-dichlorophenol, 200.0 mg (0.7 mmol) of the compound from Example 5A and 187.58 mg (1.4 mmol) of potassium carbonate are reacted according to the General Method 2.

Yield: 239 mg (84% of theory)

LC-MS (Method 2): Rₜ=3.44 min; m/z=421.0 [M+H]⁺.

¹H-NMR (400 MHz, DMSO-d₆): δ=1.35 (t, 3H), 2.27 (s, 3H), 4.39 (q, 2H), 7.42 (dd, 1H), 7.54 (dd, 1H), 7.67 (dd, 1H), 7.74-7.81 (m, 3H), 9.25 (s, 1H).

### Example 20

### 4-(2,5-Dichlorophenoxy)-2-(3-fluoro-4-methylphenyl)pyrimidine-5-carboxylic acid

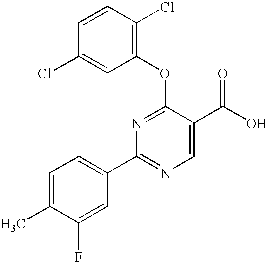

0.29 ml (0.9 mmol) of 1 N aqueous sodium hydroxide solution is added to 100.0 mg (0.2 mmol) of the compound from Example 19 in 4 ml of ethanol. The solution is stirred at room temperature overnight and then concentrated. The residue is taken up in water and acidified with 1 N hydrochloric acid, and the mixture is then extracted twice with in each case 5 ml of ethyl acetate. The organic phases are dried over magnesium sulphate and the solvent is removed under reduced pressure. The residue is purified by preparative HPLC (YMC Gel ODS-AQ S-11 μm column; mobile phase: water/acetonitrile, gradient 90:10→5:95). This gives 59 mg (63% of theory) of the product.

LC-MS (Method 1): Rₜ=2.62 min; m/z=393.1 [M+H]⁺.

¹H-NMR (300 MHz, DMSO-d,): δ=2.27 (s, 3H), 7.41 (dd, 1H), 7.53 (dd, 1H), 7.65 (dd, 1H), 7.72-7.80 (m, 3H), 9.22 (s, 1H).

### Example 21

### Ethyl 2-[3,5-di(trifluoromethyl)phenyl]-4-(2-chlorophenoxy)pyrimidine-5-carboxylate

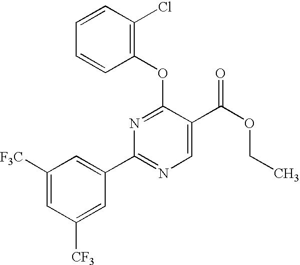

77.39 mg (0.6 mmol) of 2-chlorophenol, 200.0 mg (0.5 mmol) of the compound from Example 7A and 138.68 mg (1.0 mmol) of potassium carbonat are reacted according to the General Method 2.

Yield: 215 mg (87% of theory)

LC-MS (Method 3): Rₜ=3.32 min; m/z=491.1 [M+H]⁺.

¹H-NMR (300 MHz, DMSO-d₆): δ=1.37 (t, 3H), 4.43 (q, 2H), 7.43-7.49 (m, 1H), 7.51-7.58 (m, 2H), 7.72 (dd, 1H), 8.36 (s, 1H), 8.51 (s, 2H), 9.33 (s, 1H).

### Example 22

### 2-[3,5-Di(trifluoromethyl)phenyl]-4-(2-chlorophenoxy)pyrimidine-5-carboxylic acid

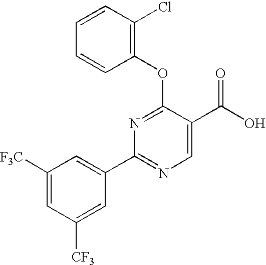

The title compound is prepared starting from Example 21 by a reaction sequence analogous to the one described in Example 18.

LC-MS (Method 1): Rₜ=2.84 min; m/z=463.0 [M+H]⁺.

¹H-NMR (300 MHz, DMSO-d₆): δ=7.40-7.50 (m, 1H), 7.51-7.56 (m, 2H), 7.71 (d, 1H), 8.35 (s, 1H), 8.50 (s, 2H), 9.29 (s, 1H).

### Example 23

### Ethyl 2-[3,5-di(trifluoromethyl)phenyl]-4-(2,5-dichlorophenoxy)pyrimidine-5-carboxylate

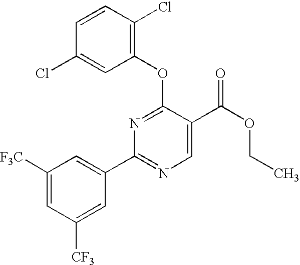

98.12 mg (0.6 mmol) of 2,5-dichlorophenol, 200.0 mg (0.5 mmol) of the compound from Example 7A and 138.66 mg (1.0 mmol) of potassium carbonate are reacted according to the General Method 2.

Yield: 216 mg (82% of theory)

LC-MS (Method 2): Rₜ=3.52 min; m/z=524.9 [M+H]⁺.

¹H-NMR (400 MHz, DMSO-d₆): δ=1.37 (t, 3H), 4.43 (q, 2H), 7.56 (dd, 1H), 7.76 (d, 1H), 7.85 (d, 1H), 8.38 (s, 1H), 8.55 (s, 2H), 9.35 (s, 1H).

### Example 24

### 2-[3,5-Di(trifluoromethyl)phenyl]-4-(2,5-dichlorophenoxy)pyrimidine-5-carboxylic acid

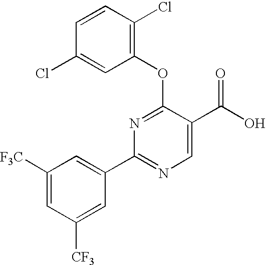

The title compound is prepared starting from Example 23 by a reaction sequence analogous to the one described in Example 18.

LC-MS (Method 1): Rₜ=2.95 min; m/z=497.0 [M+H]⁺.

¹H-NMR (300 MHz, DMSO-d₆): δ=7.56 (dd, 1H), 7.76 (d, 1H), 7.81 (d, 1H), 8.37 (s, 1H), 8.53 (s, 2H), 9.30 (s, 1H).

### Example 25

### 4-[2-Chloro-5-(trifluoromethyl)phenoxy]-2-phenylpyrimidine-5-carboxylic acid

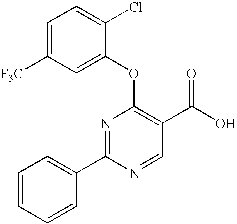

The title compound is prepared starting from Example 15 by a reaction sequence analogous to the one described in Example 18.

LC-MS (Method 2): Rₜ=2.80 min; m/z=394.9 [M+H]⁺.

¹H-NMR (300 MHz, DMSO-d₆): δ=7.42-7.58 (m, 3H), 7.77 (d, 1H), 7.92 (d, 1H), 8.03 (dd, 1H), 8.19 (d, 1H), 9.24 (s, 1H).

### Example 26

### 4-(5-Chloro-2-methylphenoxy)-2-phenylpyrimidine-5-carboxylic acid

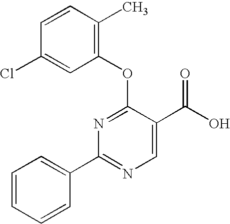

The title compound is prepared starting from Example 16 by a reaction sequence analogous to the one described in Example 18.

LC-MS (Method 2): Rₜ=2.72 min; m/z=341.0 [M+H]⁺.

¹H-NMR (300 MHz, DMSO-d₆): δ=7.35 (dd, 1H), 7.42-7.57 (m, 5H), 8.06 (dd, 2H), 9.18 (s, 1H).

### Example 27

### Ethyl 4-(2-chlorophenoxy)-2-[3-fluor-4-(trifluoromethyl)phenyl]pyrimidine-5-carboxylate

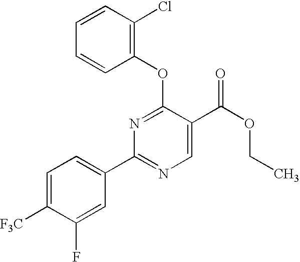

113.22 mg (0.9 mmol) of 2-chlorophenol, 300.0 mg (0.7 mmol) of the compound from Example 10A and 202.86 mg (1.5 mmol) of potassium carbonate are reacted according to the General Method 2.

Yield: 269 mg (83% of theory)

LC-MS (Method 2): Rₜ=3.41 min; m/z=440.9 [M+H]⁺.

¹H-NMR (300 MHz, DMSO-d₆): δ=1.37 (t, 3H), 4.41 (q, 2H), 7.41-7.50 (m, 1H), 7.53 (d, 2H), 7.71 (d, 1H), 7.88-8.04 (m, 3H), 9.31 (s, 1H).

### Example 28

### 4-(2-Chlorophenoxy)-2-[3-fluor-4-(trifluoromethyl)phenyl]pyrimidine-5-carboxylic acid

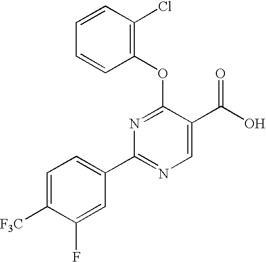

The title compound is prepared starting from Example 27 by a reaction sequence analogous to the one described in Example 18.

LC-MS (Method 3): Rₜ=2.80 min; m/z=413.2 [M+H]⁺.

¹H-NMR (300 MHz, DMSO-d₆): δ=7.40-7.56 (m, 3H), 7.53 (d, 2H), 7.70 (d, 1H), 7.68-8.02 (m, 3H), 9.26 (s, 1H).

### Example 29

### Ethyl 2-(4-chloro-3-methylphenyl)-4-(2-chlorophenoxy)pyrimidine-5-carboxylate

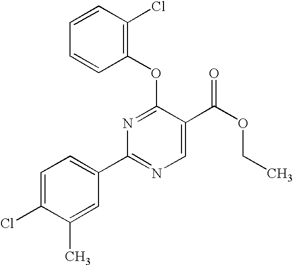

99.15 mg (0.8 mmol) of 2-chlorophenol, 200.0 mg (0.6 mmol) of the compound from Example 13A and 177.66 mg (1.3 mmol) of potassium carbonate are reacted according to the General Method 2.

Yield: 223 mg (86% of theory)

LC-MS (Method 1): Rₜ=3.23 min; m/z=403.1 [M+H]⁺.

¹H-NMR (300 MHz, DMSO-d₆): δ=1.36 (t, 3H), 2.35 (s, 3H), 4.40 (q, 2H), 7.40-7.58 (m, 4H), 7.71 (d, 1H), 7.88 (d, 1H), 7.96 (d, 1H), 9.24 (s, 1H).

### Example 30

### 2-(4-Chloro-3-methylphenyl)-4-(2-chlorophenoxy)pyrimidine-5-carboxylic acid

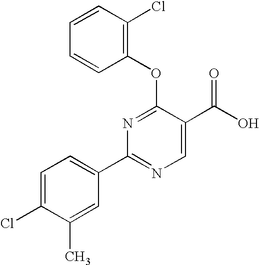

The title compound is prepared starting from Example 29 by a reaction sequence analogous to the one described in Example 18.

LC-MS (Method 3): Rₜ=2.82 min; m/z=374.9 [M+H]⁺.

¹H-NMR (400 MHz, DMSO-d₆): δ=2.35 (s, 3H), 7.40-7.55 (m, 4H), 7.70 (d, 1H), 7.86 (d, 1H), 7.95 (s, 1H), 9.20 (s, 1H).

### Example 31

### Ethyl 4-(2-chlorophenoxy)-2-(3,4-dimethylphenyl)pyrimidine-5-carboxylate

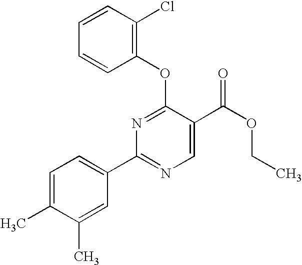

106.12 mg (0.8 mmol) of 2-chlorophenol, 200.0 mg (0.7 mmol) of the compound from Example 16A and 190.14 mg (1.4 mmol) of potassium carbonate are reacted according to the General Method 2.

Yield: 229 mg (87% of theory)

LC-MS (Method 3): Rₜ=3.27 min; m/z=383.3 [M+H]⁺.

¹H-NMR (300 MHz, DMSO-d₆): δ=1.35 (t, 3H), 2.20 (s, 3H), 2.25 (s, 3H), 4.39 (q, 2H), 7.20 (d, 1H), 7.39-7.56 (m, 3H), 7.67-7.74 (m, 2H), 7.86 (s, 1H), 9.20 (s, 1H).

### Example 32

### 4-(2-Chlorophenoxy)-2-(3,4-dimethylphenyl)pyrimidine-5-carboxylic acid

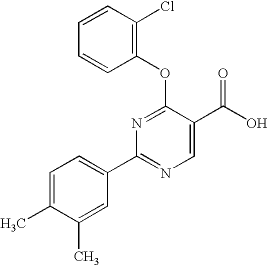

The title compound is prepared starting from Example 31 by a reaction sequence analogous to the one described in Example 18.

LC-MS (Method 3): Rₜ=2.67 min; m/z=355.2 [M+H]⁺.

¹H-NMR (400 MHz, DMSO-d₆): δ=2.20 (s, 3H), 2.24 (s, 3H), 7.19 (d, 1H), 7.38-7.59 (m, 3H), 7.69 (d, 2H), 7.85 (s, 1H), 9.16 (s, 1H).

### Example 33

### 4-(2-Chlorophenoxy)-2-(2-fluorophenyl)-pyrimidine-5-carboxylic acid

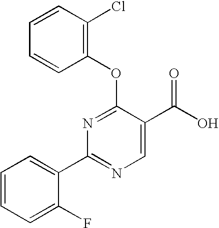

The title compound is prepared analogously to Examples 1 and 2.

¹H-NMR (300 MHz, DMSO-d₆): δ=7.22-7.31 (m, 2H), 7.32-7.41 (m, 1H), 7.42-7.49 (m, 2H), 7.50-7.59 (m, 1H), 7.54 (d, 1H), 7.77-7.87 (m, 1H), 9.21 (s, 1H), about 13.58 (br. s, 1H).

HPLC (Method 7): Rₜ=4.44 min.

MS (DCI): m/z=345.1 [M+H]⁺.

### Example 34

### 4-(2,4-Dimethylphenoxy)-2-phenylpyrimidine-5-carboxylic acid

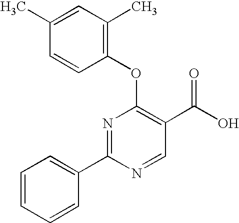

The title compound is prepared according to the General Method 3.

LC-MS (Method 10): Rₜ=2.31 min; m/z=320.1 [M]⁺.

### Example 35

### 4-(2,4-Dichloro-3,5-dimethylphenoxy)-2-phenylpyrimidine-5-carboxylic acid

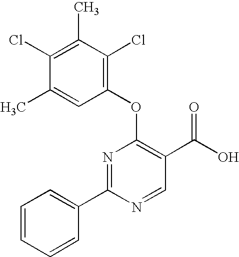

The title compound is prepared according to the General Method 3.

LC-MS (Method 10): Rₜ=2.49 min; m/z=388.0 [M]⁺.

### Example 36

### 4-(2,3-Dichlorophenoxy)-2-phenylpyrimidine-5-carboxylic acid

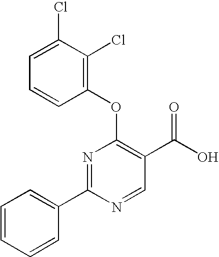

The title compound is prepared according to the General Method 3.

LC-MS (Method 10): Rₜ=2.31 min; m/z=360.0 [M]⁺.

### Example 37

### 4-(2,5-Fluorophenoxy)-2-phenylpyrimidine-5-carboxylic acid

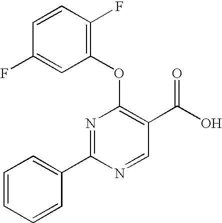

The title compound is prepared according to the General Method 3.

LC-MS (Method 10): Rₜ=2.21 min; m/z=328.0 [M]⁺.

### Example 38

### 4-(2-Chloro-4-methoxyphenoxy)-2-phenylpyrimidine-5-carboxylic acid

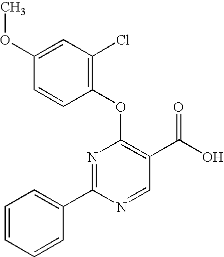

The title compound is prepared according to the General Method 3.

LC-MS (Method 10): Rₜ=2.19 min; m/z=356.0 [M]⁺.

### Example 39

### Ethyl 4-(2-cyanophenoxy)-2-phenylpyrimidine-5-carboxylate

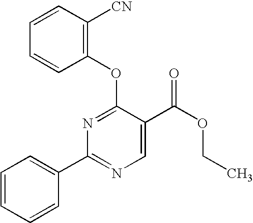

54.42 mg (0.5 mmol) of 2-hydroxybenzonitrile, 100.0 mg (0.4 mmol) of the compound from Example 2A and 105.22 mg (0.8 mmol) of potassium carbonate are reacted according to the General Method 2.

Yield: 124 mg (94% of theory)

LC-MS (Method 2): Rₜ=2.80 min; m/z=346.0 [M+H]⁺.

¹H-NMR (400 MHz, DMSO-d₆): δ1.36 (t, 3H), 4.41 (q, 2H), 7.52 (m, 2H), 7.53-7.62 (m, 2H), 7.66 (d, 1H), 7.87-7.94 (m, 1H), 8.04-8.12 (m, 3H), 9.29 (s, 1H).

### Example 40

### 4-(2-Cyanophenoxy)-2-phenylpyrimidine-5-carboxylic acid

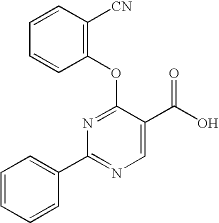

The title compound is prepared starting from Example 39 by a reaction sequence analogous to the one described in Example 18.

LC-MS (Method 2): Rₜ=2.25 min; m/z=318.1 [M+H]⁺.

¹H-NMR (400 MHz, DMSO-d₆): δ=7.44-7.50 (m, 2H), 7.52-7.56 (m, 3H), 7.87-7.93 (m, 1H), 8.04-8.09 (m, 3H), 9.27 (s, 1H).

### Example 41

### Ethyl 4-(5-cyano-2-methylphenoxy)-2-phenylpyrimidine-5-carboxylate

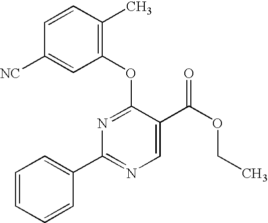

76.03 mg (0.6 mmol) of 3-hydroxy-4-methylbenzonitrile, 150.0 mg (0.6 mmol) of the compound from Example 2A and 157.83 mg (1.1 mmol) of potassium carbonate are reacted according to the General Method 2.

Yield: 180 mg (88% of theory)

LC-MS (Method 2): Rₜ=2.97 min; m/z=360.1 [M+H]⁺.

¹H-NMR (400 MHz, DMSO-d₆): δ=1.36 (t, 3H), 2.24 (s, 3H), 4.40 (q, 2H), 7.44-7.58 (m, 3H), 7.65 (d, 1H), 7.78 (d, 1H), 7.90 (s, 1H), 8.06 (d, 2H), 9.23 (s, 1H).

### Example 42

### 4-(5-Cyano-2-methylphenoxy)-2-phenylpyrimidine-5-carboxylic acid

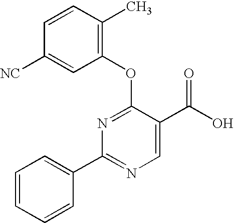

The title compound is prepared starting from Example 41 by a reaction sequence analogous to the one described in Example 18.

LC-MS (Method 3): Rₜ=2.31 min; m/z=332.2 [M+H]⁺.

¹H-NMR (400 MHz, DMSO-d₆): δ=2.24 (s, 3H), 7.43-7.57 (m, 3H), 7.64 (d, 1H), 7.77 (d, 1H), 7.88 (s, 1H), 8.04 (d, 2H), 9.22 (s, 1H).

### Example 43

### Ethyl 4-(2-chlorophenoxy)-2-(4-methyl-3-nitrophenyl)pyrimidine-5-carboxylate

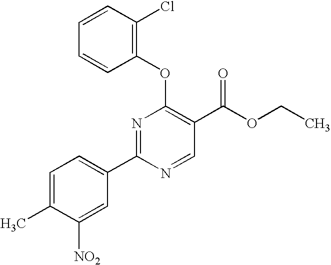

513.08 mg (4.0 mmol) of 2-chlorophenol, 1.07 g (3.3 mmol) of the compound from Example 20A and 919.30 mg (6.65 mmol) of potassium carbonate are reacted according to the General Method 2.

Yield: 1.02 g (74% of theory)

LC-MS (Method 2): Rₜ=3.18 min; m/z=414.1 [M+H]⁺.

### Example 44

### 4-(2-Chlorophenoxy)-2-(4-methyl-3-nitrophenyl)pyrimidine-5-carboxylic acid

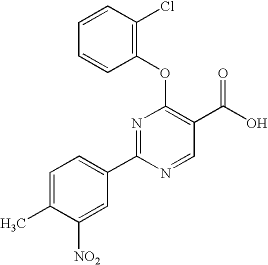

The title compound is prepared starting from Example 43 by a reaction sequence analogous to the one described in Example 18.

LC-MS (Method 1): Rₜ=2.37 min; m/z=386.1 [M+H]⁺.

¹H-NMR (400 MHz, DMSO-d₆): δ=2.56 (s, 3H), 7.41-7.48 (m, 1H), 7.50-7.56 (m, 2H), 7.62 (d, 1H), 7.70 (d, 1H), 8.16 (dd, 1H), 9.26 (s, 1H).

### Example 45

### Ethyl 4-(2-chlorophenoxy)-2-(4-fluoro-3-methoxyphenyl)pyrimidine-5-carboxylate

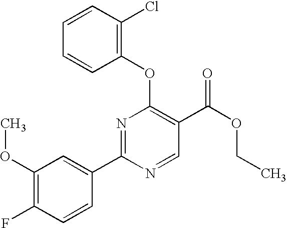

138.52 mg (1.1 mmol) of 2-chlorophenol, 279.0 mg (0.9 mmol) of the compound from Example 23A and 248.20 mg (1.8 mmol) of potassium carbonate are reacted according to the General Method 2.

Yield: 294 mg (81% of theory)

LC-MS (Method 3): Rₜ=3.10 min; m/z=403.2 [M+H]⁺.

### Example 46

### 4-(2-Chlorophenoxy)-2-(4-fluoro-3-methoxyphenyl)pyrimidine-5-carboxylic acid

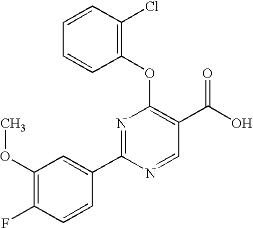

The title compound is prepared starting from Example 45 by a reaction sequence analogous to the one described in Example 18.

LC-MS (Method 2): Rₜ=2.52 min; m/z=375.1 [M+H]⁺.

¹H-NMR (400 MHz, DMSO-d₆): δ=3.75 (s, 3H), 7.31 (dd, 1H), 7.38-7.46 (m, 1H), 7.48-7.55 (m, 2H), 7.58-7.65 (m, 1H), 7.70 (d, 1H), 7.76 (dd, 1H), 9.21 (s, 1H).

### Example 47

### Ethyl 4-(2-chlorophenoxy)-2-(3,4,5-trifluorophenyl)pyrimidine-5-carboxylate

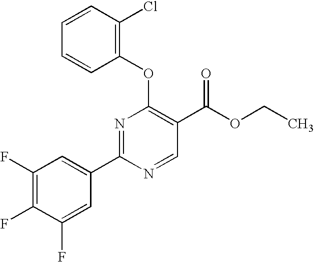

70.64 mg (0.6 mmol) of 2-chlorophenol, 145.0 mg (0.5 mmol) of the compound from Example 26A and 126.57 mg (0.9 mmol) of potassium carbonate are reacted according to the General Method 2.

Yield: 84 mg (45% of theory)

LC-MS (Method 3): Rₜ=3.31 min; m/z=409.2 [M+H]⁺.

### Example 48

### 4-(2-Chlorophenoxy)-2-(3,4,5-trifluorophenyl)pyrimidine-5-carboxylic acid

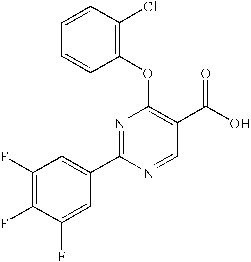

The title compound is prepared starting from Example 47 by a reaction sequence analogous to the one described in Example 18.

LC-MS (Method 2): Rₜ=2.79 min; m/z=381.0 [M+H]⁺.

¹H-NMR (400 MHz, DMSO-d₆): δ=7.42-7.57 (m, 3H), 7.69-7.78 (m, 3H), 9.24 (s, 1H).

### Example 49

### Ethyl 4-(2-chlorophenoxy)-2-(3,4-difluorophenyl)pyrimidine-5-carboxylate

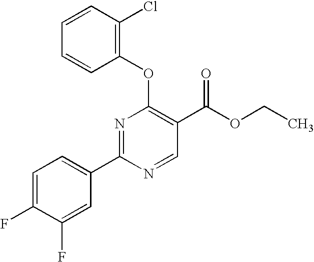

1.53 g (11.9 mmol) of 2-chlorophenol, 2.96 g (9.9 mmol) of the compound from Example 29A and 2.74 g (19.8 mmol) of potassium carbonate are reacted according to the General Method 2.

Yield: 1.75 g (45% of theory)

LC-MS (Method 3): Rₜ=3.20 min; m/z=391.3 [M+H]⁺.

### Example 50

### 4-(2-Chlorophenoxy)-2-(3,4-difluorophenyl)pyrimidine-5-carboxylic acid

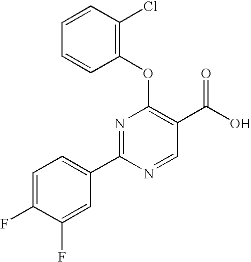

The title compound is prepared starting from Example 49 by a reaction sequence analogous to the one described in Example 18.

LC-MS (Method 3): Rₜ=2.60 min; m/z=363.1 [M+H]⁺.

¹H-NMR (400 MHz, DMSO-d₆): δ=7.41-7.45 (m, 1H), 7.47-7.61 (m, 3H), 7.71 (dd, 1H), 7.82-7.93 (m, 2H), 9.23 (s, 1H).

### Example 51

### Ethyl 4-(2-chlorobenzyl)-2-phenylpyrimidine-5-carboxylate

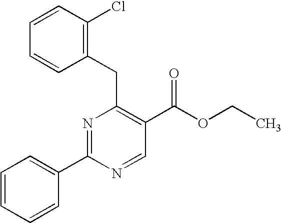

6.1 ml (about 3.0 mmol) of the (2-chlorobenzyl)zinc bromide solution in DMF from Example 30A and 87.98 mg (0.1 mmol) of tetrakis(triphenylphosphine)palladium are added to 400.0 mg (1.5 mmol) of the compound from Example 2A in 8 ml of DMF, and the mixture is stirred at room temperature overnight. Purification by preparative RP-HPLC (YMC Gel ODS-AQ S-11 μm column; mobile phase: water/acetonitrile, gradient 90:10→5:95) gives 444.0 mg (83% of theory) of the title compound.

LC-MS (Method 1): Rₜ=3.09 min; m/z=353.1 [M+H]⁺.

¹H-NMR (400 MHz, DMSO-d₆): δ=1.32 (t, 3H), 4.37 (q, 2H), 4.67 (s, 2H), 7.28-7.36 (m, 3H), 7.45-7.58 (m, 4H), 8.24 (d, 2H), 9.25 (s, 1H).

### Example 52

### 4-(2-Chlorobenzyl)-2-phenylpyrimidine-5-carboxylic acid

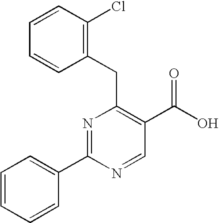

425.0 μl (0.4 mmol) of 1 N aqueous sodium hydroxide solution are added to 100.0 mg (0.3 mmol) of the compound from Example 51 in 2 ml THF. The solution is stirred at room temperature overnight and concentrated. After acidification with 1 N hydrochloric acid, the solution is extracted twice with ethyl acetate. The combined organic phases are washed with 5 ml of saturated sodium chloride solution and dried over sodium sulphate, and the solvent is removed under reduced pressure. This gives 85 mg (92% of theory) of the title compound.

LC-MS (Method 2): Rₜ=2.68 min; m/z=325.2 [M+H]⁺.

¹H-NMR (400 MHz, DMSO-d₆): δ=4.71 (s, 2H), 7.30-7.35 (m, 3H), 7.45-7.56 (m, 4H), 8.23 (d, 2H), 9.25 (s, 1H).

B. Assessment of the Pharmacological Activity

The pharmacological activity of the compounds according to the invention can be demonstrated by the following assays:

1. Cellular Transactivation Assay:

5 a) Test Principle:

A cellular assay is used to identify activators of the peroxysome proliferator-activated receptor alpha (PPAR-alpha).

Since mammalian cells contain different endogenous nuclear receptors which may complicate an unambiguous interpretation of the results, an established chimera system is used in which the ligand binding domain of the human PPARα receptor is fused to the DNA binding domain of the yeast transcription factor GAL4. The resulting GAL4-PPARα chimera is co-transfected and stably expressed in CHO cells having a reporter construct.

b) Cloning:

The GAL4-PPARα expression construct contains the ligand binding domain of PPARα (amino acids 167-468) which is PCR-amplified and cloned into the vector pcDNA3.1. This vector already contains the GAL4 DNA binding domain (amino acids 1-147) of the vector pFC2-dbd (Stratagene). The reporter construct, which contains five copies of the GAL4 binding site upstream of a thymidine kinase promoter, expresses firefly luciferase (Photinus pyralis) following activation and binding of GAL4-PPARα.

20 c) Transactivation Assay (Luciferase Reporter):

CHO (Chinese hamster ovary) cells are sown in DMEM/F12 medium (BioWhittaker) supplemented by 10% foetal calf serum and 1% penicillin/streptomycin (GIBCO), at a cell density of 2×10³ cells per well in a 384-well plate (Greiner). The cells are cultivated at 37° C. for 48 h and then stimulated. To this end, the substances to be tested are taken up in CHO-A-SFM medium (GIBCO) supplemented by 10% foetal calf serum and 1% penicillin/streptomycin (GIBCO) and added to the cells. After a stimulation period of 24 hours, the luciferase activity is measured using a video camera. The relative light units measured give, as a function of the substance concentration, a sigmoidal stimulation curve. The EC₅₀ values are calculated using the computer programme GraphPad PRISM (Version 3.02).

In this test, the compounds according to the invention show EC₅₀ values of from 5 μM to 10 nM.

2. Fibrinogen Determination:

To determine the effect on the plasma fibrinogen concentration, male Wistar rats or NMRI mice are treated with the substance to be examined by stomach tube administration or by addition to the feed for a period of 4-9 days. Under terminal anaesthesia, citrate blood is then obtained by heart puncture. The plasma fibrinogen concentrations are determined according to the Clauss method [A. Clauss, Acta Haematol. 17, 237-46 (1957)] by measuring the thrombin time using human fibrinogen as standard.

3. Description of a Test for Finding Pharmacologically Active Substances which Increase Apoprotein Al (ApoAl) and HDL Cholesterol (HDL-C) Concentrations in the Serum of Transgenic Mice Transfected with the Human ApoAl gene (hApoAl) and/or Lower Serum Triglycerides (TG):

The substances to be examined in vivo for their HDL-C-increasing activity are administered orally to male transgenic hApoAl mice. One day prior to the start of the experiment, the animals are randomized into groups with the same number of animals, generally n=7-10. Throughout the experiment, the animals have drinking water and feed ad libitum. The substances are administered orally once a day for 7 days. To this end, the test substances are dissolved in a solution of Solutol HS 15+ethanol+saline (0.9%) in a ratio of 1+1+8 or in a solution of Solutol HS 15+saline (0.9%) in a ratio of 2+8. The dissolved substances are administered in a volume of 10 ml/kg of body weight using a stomach tube. Animals which have been treated in exactly the same manner but have only been given the solvent (10 ml/kg of body weight), without test substance, serve as control group.

Prior to the first administration of substance, a blood sample from each of the mice is taken by puncture of the retroorbital venous plexus, to determine ApoAl, serum cholesterol, HDL-C and serum triglycerides (TG) (zero value). Subsequently, using a stomach tube, the test substance is administered for the first time to the animals. 24 hours after the final administration of substance (on the 8^{th} day after the beginning of treatment), a blood sample from each of the animals is again taken by puncture of the retroorbital venous plexus, to determine the same parameters. The blood samples are centrifuged and, after the serum has been obtained, TG, cholesterol, HDL-C and human ApoAl are determined using a Cobas Integra 400 plus instrument (Cobas Integra, Roche Diagnostics GmbH, Mannheim, Germany) using the respective cassettes (TRIGL, CHOL2, HDL-C and APOAT). HDL-C is determined by gel filtration and post-column derivatization with MEGA cholesterol reagent (Merck KGaA) analogously to the method of Garber et al. [J. Lipid Res. 41, 1020-1026 (2000)].

The effect of the test substances on HDL-C, hApoAl and TG concentrations is determined by subtracting the value measured for the first blood sample (zero value) from the value measured for the second blood sample (after the treatment). The means of the differences of all HDL-C, hApoAl and TG values of a group are determined and compared with the mean of the differences of the control group. Statistical evaluation is carried out using Student's t-Test, after the variances have been checked for homogeneity.

Substances which increase the HDL-C of the treated animals, compared to that of the control group, in a statistically significant (p<0.05) manner by at least 20% or which lower TG in a statistically significant (p<0.05) manner by at least 25% are considered to be pharmacologically effective.

C. Working Examples of Pharmaceutical Compositions

The compounds according to the invention can be converted into pharmaceutical preparations in the following ways:

Tablet:

Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

Production:

The mixture of the compound according to the invention, lactose and starch is granulated with a 5% strength solution (m/m) of the PVP in water. The granules are dried and then mixed with the magnesium stearate for 5 minutes. This mixture is compressed using a conventional tablet press (see above for the dimensions of the tablet). A compressive force of 15 kN is used as a guideline for the compression.

Suspension which can be Administered Orally:

Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

Composition:

500 mg of the compound according to the invention, 2.5 g of polysorbate and 97 g of polyethylene glycol 400. 20 g of oral solution correspond to a single dose of 100 mg of the compound according to the invention.

Production:

The compound according to the invention is suspended in the mixture of polyethylene glycol and polysorbate with stirring. Stirring is continued until the compound according to the invention has dissolved completely.

i.v. Solution:

The compound according to the invention is, at a concentration below saturation solubility, dissolved in a physiologically acceptable solvent (for example isotonic saline, glucose solution 5% and/or PEG 400 solution 30%). The solution is subjected to sterile filtration and filled into sterile and pyrogen-free injection containers.

1. Compound of the formula (I) 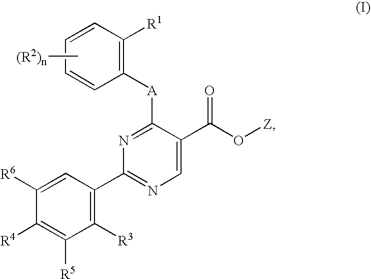
in which
A represents CH₂ or O,
R¹ represents halogen, cyano or (C₁-C₄)-alkyl,
R² represents a substituent selected from the group consisting of halogen, cyano, (C₁-C₆)-alkyl and (C₁-C₆)-alkoxy in which alkyl and alkoxy for their part may be mono- or polysubstituted by fluorine, or represents a group of the formula —NR⁷—C(═O)—R⁸, in which
R⁷ represents hydrogen or (C₁-C₆)-alkyl
and
R⁸ represents hydrogen, (C₁-C₆)-alkyl or (C₁-C₆)-alkoxy,
n represents the number 0, 1, 2 or 3,
where in the case that the substituent R² is present more than once its meanings may be identical or different,
R³ represents hydrogen, fluorine or chlorine,
R⁴ represents hydrogen, halogen, nitro, cyano, amino, trifluoromethyl, (C₁-C₄)-alkyl or (C₁-C₄)-alkoxy,
R⁵ and R⁶ are identical or different and independently of one another represent hydrogen, halogen, nitro, cyano, (C₁-C₆)-alkyl or (C₁-C₆)-alkoxy in which alkyl and alkoxy for their part may be mono- or polysubstituted by fluorine, represent amino, mono- or di-(C₁-C₆)-alkylamino, a 4- to 7-membered heterocycle which is attached via an N atom, or represent a group of the formula —NR⁹—C(═O)—R¹⁰, in which
R⁹ represents hydrogen or (C₁-C₆)-alkyl
and
R¹⁰ represents hydrogen, (C₁-C₆)-alkyl or (C₁-C₆)-alkoxy,
and
Z represents hydrogen or (C₁-C₄)-alkyl,
and its salts, solvates and solvates of the salts,
for the treatment and/or prophylaxis of diseases.

2. Compound of the formula (I) 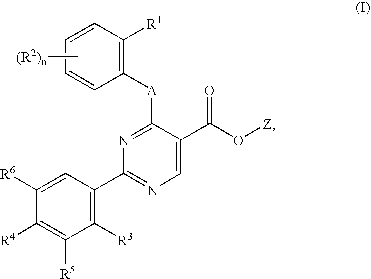
in which
A represents CH₂ or O,
R¹ represents halogen, cyano or (C₁-C₄)-alkyl,
R² represents a substituent selected from the group consisting of halogen, cyano, (C₁-C₆)-alkyl and (C₁-C₆)-alkoxy in which alkyl and alkoxy for their part may be mono- or polysubstituted by fluorine, or represents a group of the formula —NR⁷—C(═O)—R⁸, in which
R⁷ represents hydrogen or (C₁-C₆)-alkyl
and
R⁸ represents hydrogen, (C₁-C₆)-alkyl or (C₁-C₆)-alkoxy,
n represents the number 0, 1, 2 or 3,
where in the case that the substituent R² is present more than once its meanings may be identical or different,
R³ represents hydrogen, fluorine or chlorine,
R⁴ represents hydrogen, halogen, nitro, cyano, amino, trifluoromethyl, (C₁-C₄)-alkyl or (C₁-C₄)-alkoxy,
R⁵ and R⁶ are identical or different and independently of one another represent hydrogen, halogen, nitro, cyano, (C₁-C₆)-alkyl or (C₁-C₆)-alkoxy in which alkyl and alkoxy for their part may be mono- or polysubstituted by fluorine, represent amino, mono- or di-(C₁-C₆)-alkylamino, a 4- to 7-membered heterocycle which is attached via an N atom, or represent a group of the formula —NR⁹—C(═O)—R¹⁰, in which
R⁹ represents hydrogen or (C₁-C₆)-alkyl
and
R¹⁰ represents hydrogen, (C₁-C₆)-alkyl or (C₁-C₆)-alkoxy,
and
Z represents hydrogen or (C₁-C₄)-alkyl,
and its salts, solvates and solvates of the salts,
except for the compounds ethyl 4-(2-methylphenoxy)-2-phenylpyrimidine-5-carboxylate, 4-(2-methylphenoxy)-2-phenylpyrimidine-5-carboxylic acid, ethyl 4-(2,3-dimethylphenoxy)-2-phenylpyrimidine-5-carboxylate, 4-(2,3-dimethylphenoxy)-2-phenylpyrimidine-5-carboxylic acid, ethyl 2-phenyl-4-(2,4,5-trichlorophenoxy)pyrimidine-5-carboxylate and 2-phenyl-4-(2,4,5-trichlorophenoxy)pyrimidine-5-carboxylic acid.

3. Compound of the formula (I) according to claim 2, in which
A represents CH₂ or O,
R¹ represents halogen, cyano or (C₁-C₄)-alkyl,
R² represents a substituent selected from the group consisting of halogen, cyano, (C₁-C₄)-alkyl and (C₁-C₄)-alkoxy in which alkyl and alkoxy for their part may be mono- or polysubstituted by fluorine,
n represents the number 0, 1, 2 or 3,
where in the case that the substituent R² is present more than once its meanings may be identical or different,
R³ represents hydrogen, fluorine or chlorine,
R⁴ represents hydrogen, halogen, cyano, trifluoromethyl, (C₁-C₄)-alkyl or (C₁-C₄)-alkoxy,
R⁵ and R⁶ are identical or different and independently of one another represent hydrogen, halogen, nitro, cyano, (C₁-C₄)-alkyl or (C₁-C₄)-alkoxy in which alkyl and alkoxy for their part may be mono- or polysubstituted by fluorine, or represent amino, mono- or di-(C₁-C₄)-alkylamino,
and
Z represents hydrogen, methyl or ethyl,
where at least one of the radicals R³, R⁴, R⁵ and R⁶ is different from hydrogen,
and its salts, solvates and solvates of the salts.

4. Compound of the formula (I) according to claim 2 or 3 in which
A represents O,
R¹ represents fluorine, chlorine, bromine, cyano or methyl,
R² represents a substituent selected from the group consisting of fluorine, chlorine, bromine, cyano, (C₁-C₄)-alkyl, trifluoromethyl, (C₁-C₄)-alkoxy and trifluoromethoxy,
n represents the number 0, 1, 2 or 3,
where in the case that the substituent R² is present more than once its meanings may be identical or different,
R³ represents hydrogen or fluorine,
R⁴ represents hydrogen, fluorine, chlorine, trifluoromethyl or methyl,
R⁵ and R⁶ are identical or different and independently of one another represent hydrogen, fluorine, chlorine, bromine, nitro, cyano, (C₁-C₄)-alkyl, trifluoromethyl, (C₁-C₄)-alkoxy, trifluoromethoxy or amino,
and
Z represents hydrogen,
where at least one of the radicals R³, R⁴, R⁵ and R⁶ is different from hydrogen,
and its salts, solvates and solvates of the salts.

5. Process for preparing a compound of the formula (I) as defined in claims 1 to 4 in which
A represents O, characterized in that compounds of the formula (II) 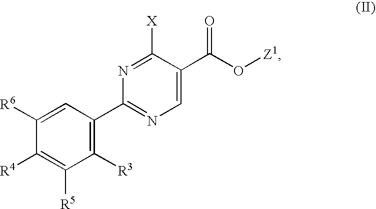
in which R³, R⁴, R⁵ and R⁶ are each as defined in claims 1 to 4 and
Z¹ represents (C₁-C₄)-alkyl
and
X represents a suitable leaving group, such as, for example, halogen, in particular chlorine,
are reacted in an inert solvent in the presence of a base with a compound of the formula (III) 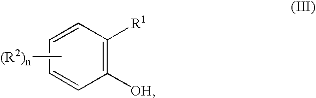
in which R¹, R² and n are each as defined in claims 1 to 4,
to give compounds of the formula (I-A) 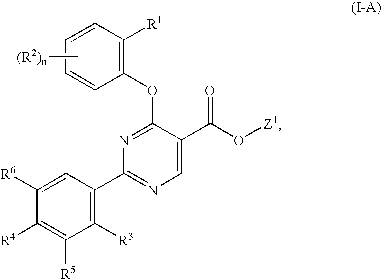
in which R¹, R², R³, R⁴, R⁵, R⁶, Z¹ and n are each as defined above,
and these are converted by basic or acidic hydrolysis into the carboxylic acids of the formula (I-B) 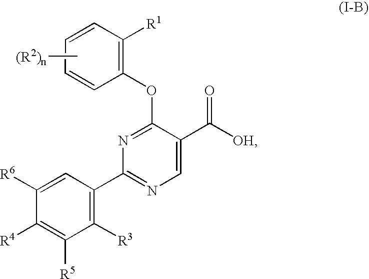
in which R¹, R², R³, R⁴, R⁵, R⁶ and n are each as defined above
and the compounds of the formulae (I-A) and (I-B) are, if appropriate, converted into their solvates, salts and/or solvates of the salts using the appropriate (i) solvents and/or (ii) bases or acids.

6. Process for preparing a compound of the formula (I) as defined in claims 1 to 3 in which A represents CH₂, characterized in that either
[A] compounds of the formula (VIII) 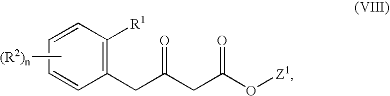
in which R¹, R² and n are each as defined in claims 1 to 3 and
Z¹ represents (C₁-C₄)-alkyl,
are reacted with a compound of the formula (IX) 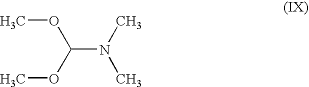
to give compounds of the formula (X) 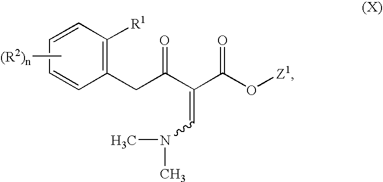
in which R¹, R², n and Z¹ are each as defined above
and then reacted in an inert solvent in the presence of a base with an amidine of the formula (V) 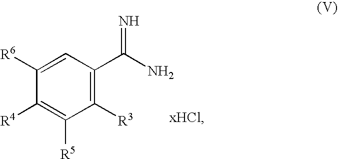
in which R³, R⁴, R⁵ and R⁶ are each as defined in claims 1 to 3,
to give compounds of the formula (I-C) 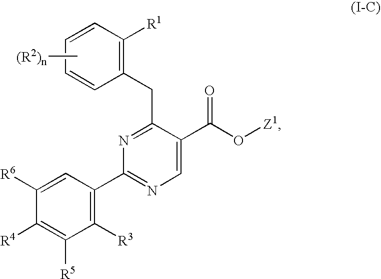
in which R¹, R², R³, R⁴, R⁵, R⁶, Z¹ and n are each as defined above
or
[B] compounds of the formula (XI) 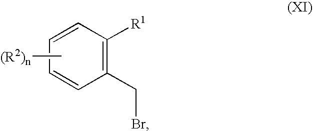
in which R¹, R² and n are each as defined in claims 1 to 3
are converted into the organotin compounds of the formula (XII) 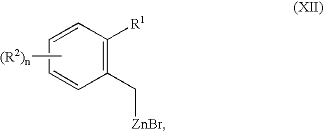
in which R¹, R² and n are each as defined above
and subsequently coupled in an inert solvent in the presence of a suitable palladium catalyst with a compound of the formula (II) 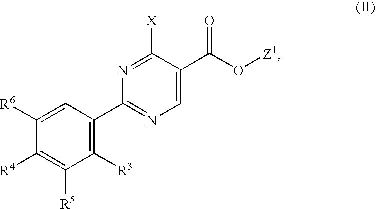
in which R³, R⁴, R⁵ and R⁶ are each as defined in claims 1 to 3 and Z¹ represents (C₁-C₄)-alkyl
and
X represents a suitable leaving group, such as, for example, halogen, in particular chlorine,
to give compounds of the formula (I-C) 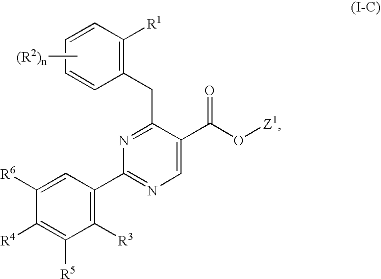
in which R¹, R², R³, R⁴, R⁵, R⁶, Z¹ and n are each as defined above
and the resulting compounds of the formula (I-C) are converted by basic or acidic hydrolysis into the carboxylic acids of the formula (I-D) 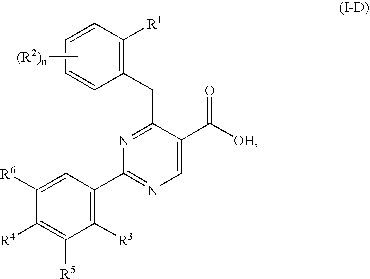
in which R¹, R², R³, R⁴, R⁵, R⁶ and n are each as defined above and the compounds of the formulae (I-C) and (I-D) are, if appropriate, converted into their solvates, salts and/or solvates of the salts using the appropriate (i) solvents and/or (ii) bases or acids.

7. Compound as defined in any of claims 2 to 4 for the treatment and/or prophylaxis of diseases.

8. Use of a compound as defined in any of claims 1 to 4 for preparing a medicament for the treatment and/or prevention of dyslipidaemias and arteriosclerosis.

9. Medicament, comprising a compound as defined in any of claims 1 to 4 in combination with an inert non-toxic pharmaceutically suitable auxiliary.

10. Medicament, comprising a compound as defined in any of claims 1 to 4 in combination with a further active compound selected from the group consisting of CETP inhibitors, HMG-CoA reductase inhibitors, squalene synthesis inhibitors, ACAT inhibitors, cholesterol absorption inhibitors, MTP inhibitors, fibrates, niacin, lipase inhibitors, PPAR-γ and/or PPAR-δ agonists, thyroid hormones and/or thyroid mimetics, polymeric bile acid adsorbers, bile acid absorption inhibitors, antioxidants, cannabinoid receptor 1 antagonists, insulin and insulin derivatives, antidiabetics, calcium antagonists, angiotensin AII antagonists, ACE inhibitors, beta-receptor blockers, alpha-receptor blockers, diuretics, platelet aggregation inhibitors and anticoagulants.

11. Medicament according to claim 9 or 10 for the treatment and/or prevent of dyslipidaemias and arteriosclerosis.

12. Method for the treatment and/or prevention of dyslipidaemias and arteriosclerosis in humans and animals by adminstering an effective amount of at least one compound as defined in any of claims 1 to 4 or of a medicament as defined in any of claims 9 to 11.
